(19) United States
(12) Patent Application Publication
(10) Pub. No.: US 2001/0056184 A1
(43) Pub. Date: Dec. 27, 2001

(54) Pipecolinic acid derivatives, method of manufacturing the same and therapeutic agents containing these compounds

(76) Inventors: Atsushi Noda, Nagano-shi (JP); Yoshinori Kobayashi, Koushoku-shi (JP); Tsuyoshi Tomiyama, Hanishina-gun (JP)

Correspondence Address: SHERMAN & SHALLOWAY, P.O. BOX 788, Alexandria, VA, 22313, US

(21) Appl. No.: 09/852,704

(22) Filed: May 11, 2001

(30) Foreign Application Priority Data
May 12, 2000 (JP) 140145/2000

(51) Int. Cl. C 07D 4 5/02; C 07D 4 1/02; C07D 211/68; C07D 211/80

(52) U.S. Cl. 546/193; 546/212; 546/216; 546/208

(57) ABSTRACT

The present invention provides a compound of the general formula (I) or a pharmaceutical salt thereof,

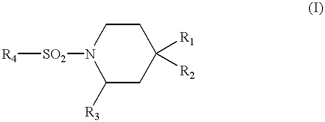

wherein, when R₁ and R₂ are the same they are represented by ═O, ═N, —OR₉ (where R₉ is hydrogen, lower alkyl or benzyl), when R₁ and R₂ are different and one of R₁ and R₂ is represented by hydrogen, the other is represented by —R₅—R₆—, (where R₅ is —O—, —NH—, —NHCO—, or —NHSO₂—, and where R₆ is hydrogen, lower alkyl, —Ph—R₇, —(CH₂)n—O—Ph—R₇, (where n=1-6, R₇ is hydrogen, hydroxy, lower alkyl, lower alkoxy, halogen, nitro, or pyridyloxy), indolyl, N-oxidepyridyl, phthalimide, thienyl, or pyridyl); R₃ represents —COOH, —COOEt, —COOMe, —CH₂N(OH)CHO, or —CONHOH; R₄ represents lower alkyl, thienyl, —Ph—R₈ (where R₉ represents hydroxy, lower alkyl, lower alkoxy, nitro, halogen, pyridyloxy, or phenyl group substituted hydrogen, lower alkyl, lower alkoxy, hydroxy, and halogen), and methods of making compounds within the class of the general formula (I).

### BACKGROUND OF THE INVENTION

1. Field of the Invention

This invention relates to the pipecolinic acid and the derivative compounds thereof which have a Matrix Metalloproteinases (hereinafter shortened to MMPs) inhibitory function or the pharmaceutically permittable salt thereof and the production method thereof, further relates to a medicine composition containing said carboxylic acid and derivative compounds thereof or the salt thereof.

2. Description of the Prior Art

The MMPs are zinc dependent, calcium requiring enzymes that are involved in the degradation of extra cellular matrix. Under normal physiological conditions, the expression of the constitutive MMPs is low, and regulated by naturally occurring inhibitors termed TIMPs (tissue inhibitor of metalloproteinases). However, under pathological conditions such as rheumatoid and osteoarthritis, MMPs expression in cartilage is disregulated and resulted in over expression of MMPs which are not controlled by constitutive TIMPs. The level of the MMPs are high with enzymic activity and exceeding the level of the TIMPs. This condition leads to a loss of proteoglycan and collagen (J. Trzaskos, et al., Acta. Onthopaedica Scandinavia, 66, 150 (1995)).

In addition, MMPs inhibitors are effective on treatment for corneal ulceration and tumor progression (R. P.Beckett et al., D.D.T., 1, 16 (1996)), and MMPs are playing important role in the pathogenesis of arteriosclerosis and restenosis after percutaneous transluminal coronary angioplasty (PTCA) (C. M. Dollery et al., Circ. Res.,77, 863 (1995)). Furthermore, application of MMPs inhibitors are effecitve on treatment for inflammatory bowel disease (Sylvia L., F. Pender et al., J. Immunol. ,158, 1582 (1997)). It is therapeutically useful to control the increased MMPs by MMPs inhibitors under these pathological conditions. Recently, a lot of MMPs inhibitors have been reported (R.Paul Beckett et al., Exp.Opin.Ther.Patents, 8 (3), 259-282 (1996)).

3. Problems to be Solved by the Invention

Though many reported MMPs inhibitors had excellent in vitro activity, these compounds had poor oral bioavailabilities. For example, the compounds had been performed intrapleural administration (Drug News & Perspectives, 8 (4), 247 (1995)) or eye drops (Drug of the Future, 18, 1101 (1993)).

The object of the present invention is the provision of pharmaceutical compositions useful as non-piptidic MMPs inhibitors being able to oral administration and a production method thereof.

### DETAILED DESCRIPTION OF THE INVENTION

The present invention provides a new pipecolinic acid derivative compound of general formula (I):

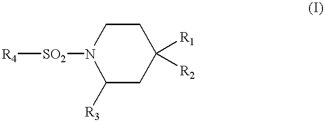

and its salt capable of being used for medical treatment. The lower alkyl mentioned in general formula (I) represents the straight or branched C₁-C₆ alkyl group, so as methyl, ethyl, propyl, butyl, pentyl, hexyl, isopropyl, isobutyl, isopentyl, cyclopropyl, cyclohexyl etc. The lower alkoxy represents alkoxy groups containing C₁- C₆ carbon atom. The compound of general formula (I) contain several isomers, so this invention contains these isomers.

In the compound of general formula (I), pharmacologically, R₃ prefers —COOH and CONHOH. R₁ and R₂ prefer that one of two groups is hydrogen and the other is —R₅ -R₆.

In this case, R₅ prefers —NH— and —NHCO—, and R₆ prefers phenyl group that is substituted by methyl, pyridyl, methoxy, halogen, or nitro. R₄ prefers phenyl and biphenyl which are substituted by methoxy, halogen, or nitro, respectively. Furthermore, the compound which containing phenyl groups exhibit non-selective inhibition, and the compound which containing biphenyl groups exhibit selective inhibition for MMP-2 and MMP-9.

In the isomer, (2R, 4R)-configuration is preferred.

The compound of general formula (I) can be obtained as follows. 4-Hydroxy-pipecolinic acid methyl ester is used as starting material, and the compound has (2R, 4S)- and (2S, 4R)- isomer. Both isomers are used for the preparetion of the compound of general formula (I), and the isomer of general formula (I) can be obtained.

(A) In the case of R₁ is hydrogen atom, R₂ is —OH, and R₃ is —COOH in the compounds of general formula (I), it is prepared by the following reactions.

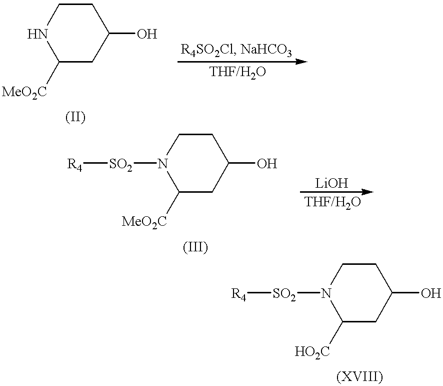

(wherein R₄ is the same as mentioned above.)

4-hydroxypipecolinic acid (II) is reacted with sulfonyl chloride using organic base for example triethylamine, pyridine, etc, or mineral base for example sodium carbonate, potassium carbonate, etc and THF/H₂O, etc as a mixed solvent to yield compound (III). Then the desired compound (XVIII) is obtained by hydrolysis of the compound (III) using alkali - metal hydroxide (NaOH, KOH or LiOH).

(B) In case of R₁ is hydrogen atom, R₂ is —OH, and R₃ is —CONHOH in the compounds of general formula (I), it is prepared by the following reactions.

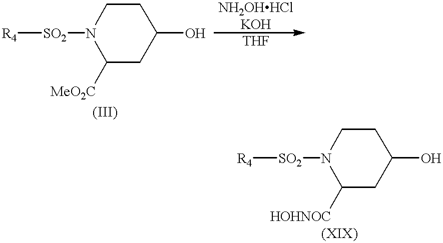

(wherein R₄ is the same as mentioned above.)

The desired compound (XIX) is obtained by the reaction of the compound (III) and hydroxylamine hydrochloride salt using potassium hydroxide in THF.

(C) In the case of R₁.and.R₂ are ═O, R₃ is —COOH in the compounds of the general formula (I), it is prepared by the following reactions.

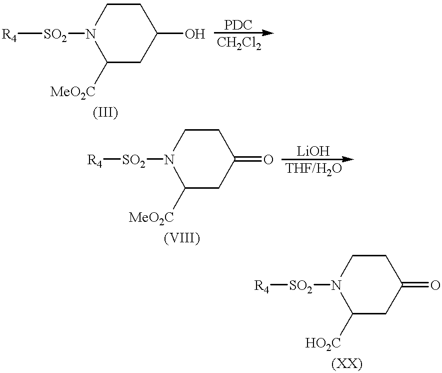

(wherein R₄ is the same as mentioned above.)

The compound of general formula (III) is reacted with oxidant for example PDC, PCC, etc in CH₂Cl₂, DMF, etc as a solvent to yield the compound of general formula (VIII).

The desired compound of general formula (XX) is obtained by alkali hydrolisys of that compound.

(D) In case of R₁ and R₂ are ═O, and R₃ is —CONHOH in the compounds of the general formula (I), it is prepared by the following reactions.

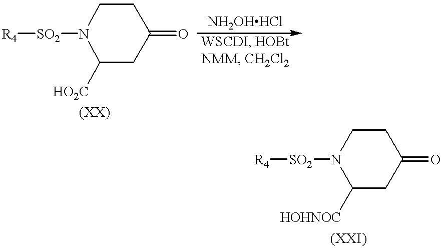

(wherein R₄ is the same as mentioned above.)

The compound of the general formula (XX) is reacted with hydroxylamine hydrochloride salt using coupling reagents, for example WSCDI (1-(3-dimethylaminopropyl)-3-ethylcarbodiimide . HCl salt), DCC (1,3-dicyclohexylcarbodiimide), DPPA (diphenylphosphorylazide), DEPC (diethylphosphoryl cyanide), etc, and in the presence of the base, for example NMM (N-methylmorpholine), triethylamine, etc, in methlenechloride, THF, DMF, etc as a soluvent, to yield the desired compound of the general formula (XXI).

(E) In case of R₁ is hydrogen atom, R₂ is —NH₂, and R₃ is —COOH in the compounds of the general formula (I), it is prepared by the following reactions.

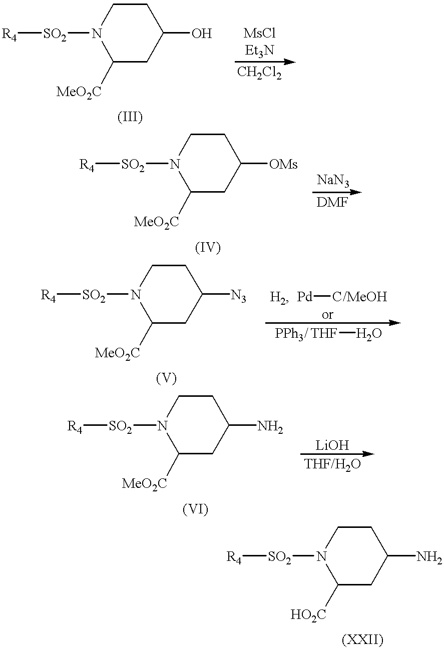

(wherein R₄ is the same as mentioned above.)

The compound of general formula (III) is reacted with MsCl (methanesulfonyl chloride) or TsCl (p-toluenesulfonyl chloride ) in the precense of the base for example triethylamine, pyridine, etc, in THF, CH₂Cl₂, DMF, etc as a solvent to yield the compound of general formula (IV), then it is reacted with sodium azide in THF, DMF, etc as a solvent to yield the compound of general formula (V). Subsequently, The compound of general formula (V) is converted to the compound of general formula (VI) by hydrogenation with Pd in methnol, THF, etc as a solvent under H₂ atomosphere, or, with triphenylphosphine in THF/H₂O mixed solvent, then it is hydrolyzed to yield the compound of general formula (XXII).

(F) In case of R₁ is hydrogen atom, R₂ is —NH₂, and R₃ is —CONHOH in the compounds of the general formula (I), it is prepared by the following reactions.

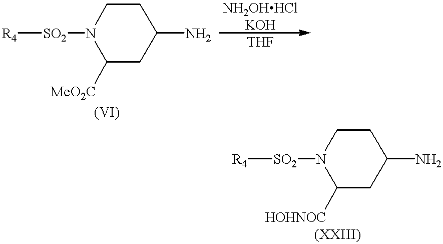

(wherein R₄ is the same as mentioned above.)

The compound of the general formula (VI) is reacted with the THF solution that disolved hydroxylamine hydrochloride salt and potassium hydroxide in THF solution to yield the desired compound of the general formula (XXIII).

(G) In case of R₁ is hydrogen atom, R₂ is —OH, and R₃ is —COOH in the compounds of the general formula (I)which is isomer of the compound on preparetion of method (A), it is prepared by the following reactions.

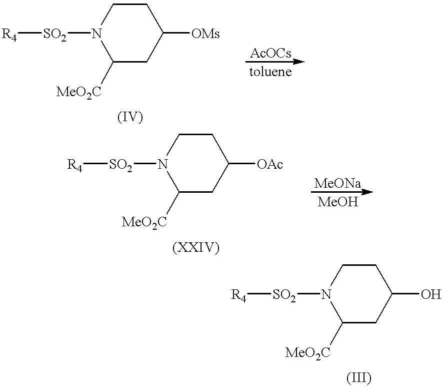

(wherein R₄ is the same as mentioned above.)

The compound of the general formula (IV) is reacted with cesium acetate in THF, toluene, etc as a soluvent to yield the compound of the general formula (XXIV), then it is reacted with sodium methoxide in methanol to yield the desired compound of the general formula (III).

(H) In case of R₁ is hydrogen atom, R₂ is —NHCO—R_{6,} and R₃ is —COOH in the compounds of the general formula (I), it is prepared by the following reactions.

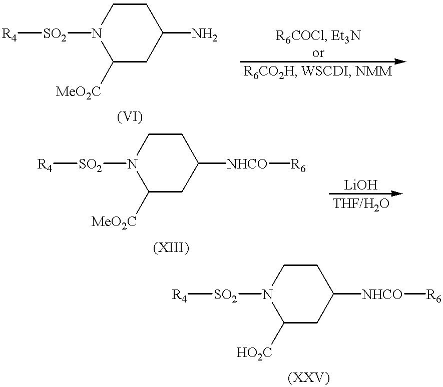

(wherein R₄ and R₆ are the same as mentioned above.)

The compound of the general formula (VI) is reacted with acid chloride in the presence of organic base, for example triethylamine, pyridine, etc. or mineral base, for example sodium carbonate, potassium carbonate, etc in CH₂Cl₂, CHCl₃, etc as a solvent to yield the compound of the general formula (XIII). Then it is hydrolyzed with lithium hydroxide to yield the desired compound of the general formula (XXV).

(I) In case of R₁ is hydrogen atom, R₂ is —NHCO—R₆, R₃ is —COOH, and R₄ is —Ph—R₈ in the compounds of the general formula (I), it is prepared by the following reactions.

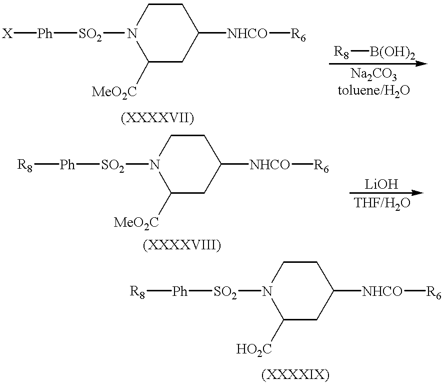

(wherein R₆ is the same as mentioned above, R₈ is H, lower alkyl, lower alcoxy, hydroxy, or halogen substituted phenyl group, X is halogen)

The compound of the general formula (XXXXVII) is subjected to cross-coupling reaction with a boronic acid using palladium or nickel catalyst to yield the compound of the general formula (XXXXVIII) (A. Suzuki, Synth. Org. Chem. Jpn., 1988, 46, 848.; E. Negishi, J. Org. Chem., 1977, 42, 1821.; J. K. Stille, J. Org. Chem., 1987, 52, 422.). Tetrakis (triphenylphosphine) palladium, palladium chloride, palladium acetate, etc is used as the catalyst, and the reaction carried out in the presence of the base, for example sodium carbonate, potassium carbonate, cesium carbonate, etc in toluene, DMF, or H₂O. Then the compound of general formula (XXXXVIII) is hydrolyzed with lithium hydroxide to yield the desired compound of the general formula (XXXXIX).

(J) In case of R₁ is hydrogen atom, R₂ is —NHCO—R₆, and R₃ is —CONHOH in the compounds of the general formula (I), it is prepared by the following reactions.

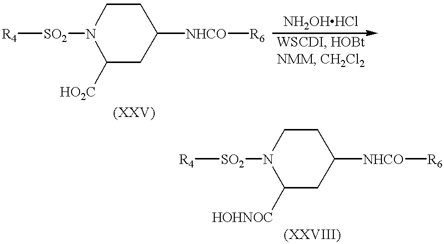

(wherein R₄ and R₆ are the same as mentioned above.)

The compound of the general formula (XXV) is reacted with hydroxylamine hydrochloride salt using coupling reagents, for example WSCDI, DCC, DPPA, DEPC, etc, and in the presence of the base, for example NMM (N - methylmorpholine), triethylamine, etc, in methlenechloride, THF, DMF, etc as a soluvent, to yield the desired compound of the general formula (XXVIII).

The other method, the compound of the general formula (XXV) is condenced with O-benzylhydroxylamine hydrochloride salt instead of hydroxylamine hydrochloride salt using tha same condition, followed by debenzylation with palladium on carbone to yield the desired compound of the general formula (XXVIII).

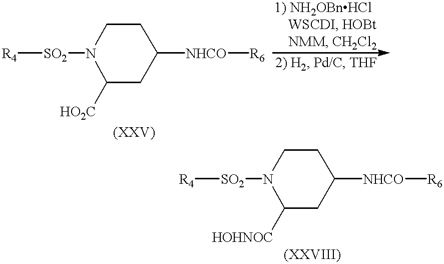

(wherein R₄ and R₆ are the same as mentioned above.)

(K) In case of R₁ is hydrogen atom, R₂ is —NHSO₂—R₆, and R₃ is —COOH in the compounds of the general formula (I), it is prepared by the following reactions.

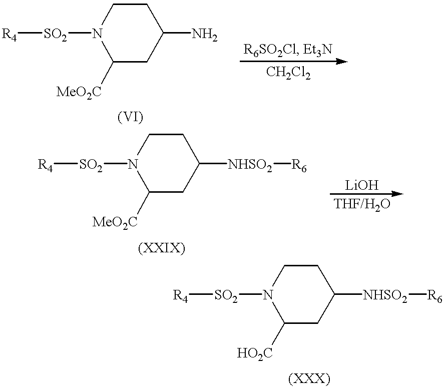

(wherein R₄ and R₆ are the same as mentioned above.)

The compound of the general formula (VI) is reacted with sulfonyl chloride in the presence of organic base, for example triethylamine, pyridine, etc. or mineral base, for example sodium carbonate, potassium carbonate, etc in CH₂Cl₂, CHCl₃, etc as a solvent to yield the compound of the general formula (XXIX). Then it is hydrolyzed with lithium hydroxide to yield the desired compound of the general formula (XXX).

(L) In case of R₁ is hydrogen atom, R₂ is —NHSO₂—R₆, and R₃ is —CONHOH in the compounds of the general formula (I), it is prepared by the following reactions.

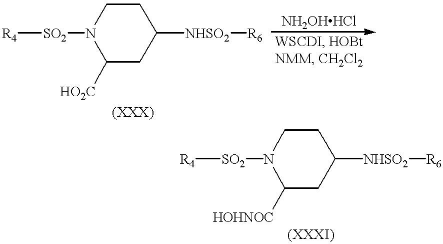

(wherein R₄ and R₆ are the same as mentioned above.)

The compound of the general formula (XXX) is reacted with hydroxylamine hydrochloride salt using coupling reagents, for example WSCDI, DCC, DPPA, DEPC, etc, and in the presence of the base, for example NMM (N-methylmorpholine), triethylamine, etc, in methlenechloride, THF, DMF, etc as a soluvent, to yield the desired compound of the general formula (XXXI).

The other method, the compound of the general formula (XXX) is condenced with O-benzylhydroxylamine hydrochloride salt instead of hydroxylamine hydrochloride salt using tha same condition, then it is debenzylated with palladium on carbone to yield the desired compound of the general formula (XXXI).

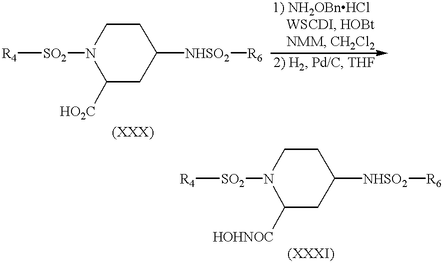

(wherein R₄ and R₆ are the same as mentioned above.)

(M) In case of R₁ is hydrogen atom, R₂ is —NH—R₆, and R₃ is —COOH in the compounds of the general formula (I), it is prepared by the following reactions.

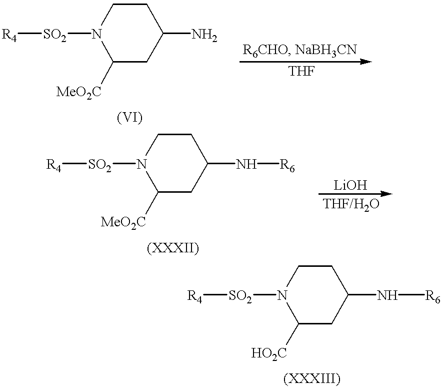

(wherein R₄ and R₆ are the same as mentioned above.)

The compound of the general formula (VI) is reacted with aldehyde using sodium borohydride, cyano borohydride, or palladium on carbone in methanol, THF, etc as a soluvent to yield the compound of the general formula (XXXII). Then it is hydrolyzed with lithium hydroxide to yield the desired compound of the general formula (XXXIII).

(N) In case of R₁ is hydrogen atom, R₂ is —NH—R₆, and R₃ is —CONHOH in the compounds of the general formula (I), it is prepared by the following reactions.

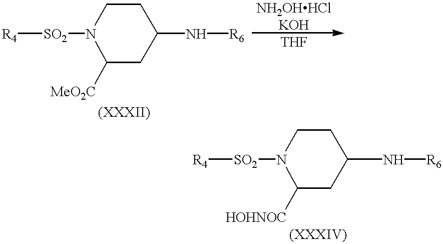

(wherein R₄ and R₆ are the same as mentioned above.)

The compound of the general formula (XXXII) is reacted with hydroxylamine hydrochloride salt and potassium hydroxide in THF solution, to yield the desired compound of the general formula (XXXIV).

(O) In case of R₁ and R₂ are ═N—OR₉, R₃ is —COOH in the compounds of the general formula (I), it is prepared by the following reactions.

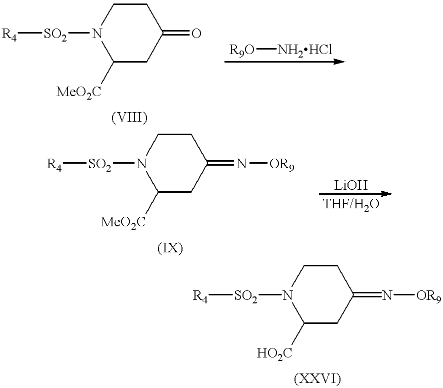

(wherein R₄ and R₉ are the same as mentioned above.)

The compound of the general formula (VIII) is reacted with R₉O—NH₂ HCl (R₉ is methyl or benzyl), in the presence of the base, for example triethylamine, etc to yield the compound of the general formula (IX). Then it is hydrolyzed with lithium hydroxide to yield the desired compound of the general formula (XXVI).

(P) In case of R₁ and R₂ are ═N—OR₉, R₃ is —CONHOH in the compounds of the general formula (I), it is prepared by the following reactions.

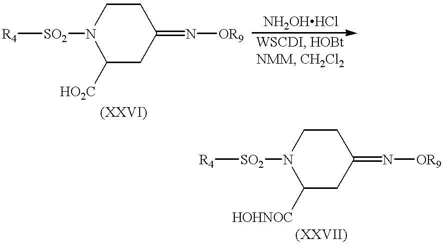

The compound of the general formula (XXVI) is reacted with hydroxylamine hydrochloride salt using coupling reagents, for example WSCDI, DCC, DPPA, DEPC, etc, and in the presence of the base, for example NMM (N-methylmorpholine), triethylamine, etc, in methlenechloride, THF, DMF, etc as a soluvent, to yield the desired compound of the general formula (XXVII).

(Q) In case of R₁ is hydrogen atom, R₂ is —NHCO—R₆, and R₃ is —CH₂N(OH)COH in the compounds of the general formula (I), it is prepared by the following reactions.

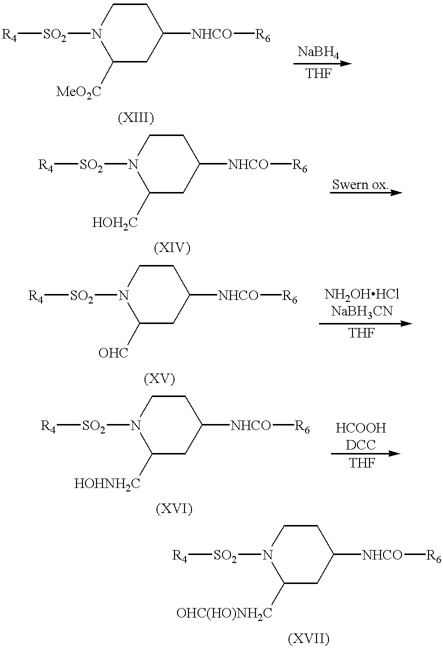

(wherein R₄ and R₆ are the same as mentioned above.)

The compound of the general formula (XIII) is reduced with lithium borohydride, etc as a reductant in THF to yield the compound of the general formula (XIV), then it is done swern oxidation to yield the compound of the general formula (XV). Subsecuently, the compound of the general formula (XV) is reacted with hydroxylamine hydrochloride salt using sodium borohydride, cyano borohydride, or palladium on carbone in methanol, THF, etc as a soluvent to yield the compound of the general formula (XVI).Furthermore, it is reacted with formic acid using coupling reagent such as DCC, WSCDI, etc in THF, etc as a solvent to yield the desired compound of the general formula (XVII).

The compounds related to the general formula (I) are exemplified as follows.

1. (2R, 4R)-2-Carboxy-1-(4-methoxybenzenesulfonyl)-4-(4-methylpentanoylamino)-piperidine (Compound 1)

2. (2R, 4R)-2-Carboxy-1-(4-methoxybenzenesulfonyl)-4-[5-(4-methoxyphenoxy) pentanoylamino)-piperidine (Compound 2)

3. (2R, 4R)-2-Carboxy-1-(4-methoxybenzenesulfonyl)-4-(4-methoxybenzoylamino)- piperidine (Compound 3)

4. (2R, 4R)-2-Carboxy-4-(4-methoxybenzoylamino)-1-[4-(4-methoxyphenyl) benzenesulfonyl)-piperidine (Compound 4)

5. (2R, 4R)-2-Carboxy-1-[4-(4-methoxyphenyl)benzenesulfonyl]-4-(4-methylpentanoylamino)-piperidine (Compound 5)

6. (2R, 4R)-2-Carboxy-4-(4-methoxybenzoylamino)-1-(2-thiophenesulfonyl)-piperidine (Compound 6)

7. (2R, 4R)-2-Carboxy-4-(4-methylpentanoylamino)-1-(2-thiophenesulfonyl)-piperidine (Compound 7)

8. (2R, 4R)-1-(4-Bromobenzenesulfonyl)-2-carboxy-4-(4-methylpentanoylamino)-piperidine (Compound 8)

9. (2R, 4R)-1-(4-Bromobenzenesulfonyl)-2-carboxy-4-(4-methoxybenzoylamino)-piperidine (Compound 9)

10. (2R, 4R)-2-Carboxy-1-[3-(4-methoxyphenyl)benzenesulfonyl]-4-(4-methylpentanoylamino)-piperidine (Compound 10)

11. (2R, 4R)-2-Carboxy-1-[3-(4-hydroxyphenyl)benzenesulfonyl]-4-(4-methylpentanoylamino)-piperidine (Compound 11)

12. (2R, 4R)-2-Carboxy-1-[2-(4-methoxyphenyl)benzenesulfonyl]-4-(4-methyl -pentanoylamino)-piperidine (Compound 12)

13. (2R, 4R)-2-Carboxy-1-[2-(4- hydroxyphenyl)benzenesulfonyl]-4-(4-methylpentanoylamino)-piperidine (Compound 13)

14. (2R, 4R)-4-Benzylamino -2-carboxy- -1-[4-(4-methoxyphenyl)benzenesulfonyl]-piperidine (Compound 14)

15. (2R, 4R)-2-Carboxy-1-[4-(4- hydroxyphenyl)benzenesulfonyl]-4-(4-methylpentanoylamino)-piperidine (Compound 15)

16. (2R, 4R)-2-Carboxy-4-(4-methoxybenzenesulfonylamino)-1-[4-(4-methoxyphenyl) benzenesulfonyl]-piperidine (Compound 16)

17. (2R, 4R)-4-Benzoylamino-2-carboxy-1-[4-(4-methoxyphenyl)benzenesulfonyl]- piperidine (Compound 17)

18. (2R, 4R)-4-Benzoylamino-2-carboxy-1-[4-(4-hydroxyphenyl)benzenesulfonyl]-piperidine (Compound 18)

19. (2R, 4R)-2-Carboxy-1-[4-(4-methoxyphenyl)benzenesulfonyl]-4-(4-methylpentylamino)-piperidine (Compound 19)

20. (2R, 4R)-2-Carboxy-4-(4-methoxybenzylamino)benzenesulfonyl-1-[4-(4-methoxyphenyl) benzenesulfonyl]-piperidine (Compound 20)

21. (2R, 4R)-2-Carboxy-1-(4-hydroxybenzenesulfonyl)-4-(4-methylpentanoylamino)-piperidine (Compound 21)

22. (2R, 4R)-4-Acetylamino-2-carboxy-1-[4-(4-methoxyphenyl)benzenesulfonyl]-piperidine (Compound 22)

23. (2R, 4R)-4-Acetylamino-2-carboxy-1-[4-(4-hydroxyphenyl)benzenesulfonyl]-piperidine (Compound 23)

24. (2R, 4R)-4-Acetylamino-2-carboxy-1-(4-methoxybenzenesulfonyl]-piperidine (Compound 24)

25. (2R, 4R)-2-Carboxy-1-(4-methoxybenzenesulfonyl)-4-phtalimidyl-piperidine (Compound 25)

26. (2R, 4R)-2-Carboxy-1-[4-(4-methoxyphenyl)benzenesulfonyl]-4-phtalimidyl-piperidine (Compound 26)

27. (2R, 4R)-2-Carboxy-4-ethylamino-1-[4-(4-methoxyphenyl)benzenesulfonyl]-piperidine (Compound 27)

28. (2R, 4R)-2-Carboxy-4-diethylamino-1-[4-(4-methoxyphenyl)benzenesulfonyl]-piperidine (Compound 28)

29. (2R, 4R)-2-Carboxy-4-(4-methylpentylamino)-1-(2-thiophenesulfonyl)-piperidine (Compound 29)

30. (2R, 4R)-2-Carboxy-1-[4-(4-chlorophenyl)benzenesulfonyl]-4-(4-methylpentanoylamino)-piperidine (Compound 30)

31. (2R, 4R)-4-Acetylamino-2-carboxy-1-[4-(4- chlorophenyl)benzenesulfonyl]-piperidine (Compound 31)

32. (2R, 4R)-2-Carboxy-1-[4-(4-methoxyphenyl)benzenesulfonyl]-4-(2-thiophenecarbonylamino)-piperidine (Compound 32)

33. (2R, 4R)-2-Carboxy-1-[4-(4-methoxyphenyl)benzenesulfonyl]-4-(2-pyridinecarbonylamino)-piperidine (Compound 33)

34. (2R, 4R)-2-Carboxy-1-[4-(4- chlorophenyl)benzenesulfonyl]-4-(2-pyridinecarbonylamino)-piperidine (Compound 34)

35. (2R, 4R)-2-Carboxy-1-[4-(4-methoxyphenyl)benzenesulfonyl]-4-(4-nitrobenzoyllamino)-piperidine (Compound 35)

36. (2R, 4R)-2-Carboxy-4-(3-indolecarbonylamino)-1-[4-(4-methoxyphenyl) benzenesulfonyl]-piperidine (Compound 36)

37. (2R, 4R)-2-Carboxy-4-(4-methylpentanoylamino)-1-(4-nitrobenzenesulfonyl)-piperidine (Compound 37)

38. (2R, 4R)-2-Carboxy-4-(4-methylpentanoylamino)-1-[4-(4-nitrophenyl) benzenesulfonyl]-piperidine (Compound 38)

39. (2R, 4R)-2-Carboxy-1-[4-(4-methoxyphenyl)benzenesulfonyl]-4-(pyridin-N-oxide-2-yl) carbonylamino-piperidine (Compound 39)

40. (2S, 4S)-2-Carboxy-1-(4-methoxybenzenesulfonyl)-4-[5-(4-methoxyphenoxy) pentanoylamino]-piperidine (Compound 40)

41. (2S, 4S)-2-Carboxy-1-(4-methoxybenzenesulfonyl)-4-(4-methylpentanoylamino)-piperidine (Compound 41)

42. (2S, 4S)-2-Carboxy-1-(4-methoxybenzenesulfonyl)-4-(4-methoxybenzoylamino)-piperidine (Compound 42)

43. (2S, 4S)-4-Acetylamino-2-carboxy-1-(4-methoxybenzenesulfonyl)-piperidine (Compound 43)

44. (2R, 4S)-4-Acetylamino-2-carboxy-1-[4-(4-methoxyphenyl)benzenesulfonyl]-piperidine (Compound 44)

45. (2R, 4R)-2- Hydroxyaminocarbonyl-1-(4-methoxybenzenesulfonyl)-4-(4-methylpentanoylamino)-piperidine (Compound 45)

46. (2R, 4R)-2- Hydroxyaminocarbonyl-1-(4-methoxybenzenesulfonyl)-4-[5-(4-methoxyphenoxy) pentanoylamino]-piperidine (Compound 46)

47. (2R, 4R)-2- Hydroxyaminocarbonyl-1-(4-methoxybenzenesulfonyl)-4-(4-methoxybenzoylamino)-piperidine (Compound 47)

48. (2R, 4R)-2- Hydroxyaminocarbonyl-4-(4-methoxybenzoylamino)-1-[4-(4-methoxyphenyl) benzenesulfonyl]-piperidine (Compound 48)

49. (2R, 4R)-2- Hydroxyaminocarbonyl-1-[4-(4-methoxyphenyl)benzenesulfonyl]-4-(4-methylpentanoylamino)-piperidine (Compound 49)

50. (2R, 4R)-2-Hydroxyaminocarbonyl-4-(4-methoxybenzoylamino)-1-(2-thiophenesulfonyl)-piperidine (Compound 50)

51. (2R, 4R)-2-Hydroxyaminocarbonyl-4-(4-methylpentanoylamino)-1-(2-thiophenesulfonyl)-piperidine (Compound 51)

52. (2R, 4R)-2-Hydroxyaminocarbonyl-1-[3-(4-methoxyphenyl)benzenesulfonyl]-4-(4-methylpentanoylamino)-piperidine (Compound 52)

53. (2R, 4R)-2-Hydroxyaminocarbonyl-1-[3-(4-hydroxyphenyl)benzenesulfonyl]-4-(4-methylpentanoylamino)-piperidine (Compound 53)

54. (2R, 4R)-2-Hydroxyaminocarbonyl-1-[2-(4-methoxyphenyl)benzenesulfonyl]-4-(4-methylpentanoylamino)-piperidine (Compound 54)

55. (2R, 4R)-2-Hydroxyaminocarbonyl-1-[2-(4-hydroxyphenyl)benzenesulfonyl]-4-(4-methylpentanoylamino)-piperidine (Compound 55)

56. (2R, 4R)-4-Benzylamino-2-hydroxyaminocarbonyl-1-[4-(4-methoxyphenyl) benzenesulfonyl]-piperidine (Compound 56)

57. (2R, 4R)-2-Hydroxyaminocarbonyl-1-[4-(4-hydroxyphenyl)benzenesulfonyl]-4-(4-methylpentanoylamino) piperidine (Compound 57)

58. (2R, 4R)-2-Hydroxyaminocarbonyl-1-methanesulfonyl -4-(pyridin -2-yl) carbonylamino-piperidine (Compound 58)

59. (2R, 4R)-4-Benzoylamino-2-hydroxyaminocarbonyl-1-[4-(4-methoxyphenyl) benzenesulfonyl]-piperidine (Compound 59)

60. (2R, 4R)-4-Benzoylamino-2-hydroxyaminocarbonyl-1-[4-(4-hydroxyphenyl) benzenesulfonyl]-piperidine (Compound 60)

61. (2R, 4R)-2-Hydroxyaminocarbonyl-1-[4-(4-methoxyphenyl)benzenesulfonyl]-4-(4-methylpentylamino)-piperidine (Compound 61)

62. (2R, 4R)-2-Hydroxyaminocarbonyl-4-(4-methoxybenzylamino)-1-[4-(4-methoxyphenyl) benzenesulfonyl]-piperidine (Compound 62)

63. (2R, 4R)-2-Hydroxyaminocarbonyl-1-[4-(4-hydroxybenzenesulfonyl)-4-(4-methylpentanoylamino)-piperidine (Compound 63)

64. (2R, 4R)-4-Acetylamino-2-hydroxyaminocarbonyl-1-[4-(4-methoxyphenyl) benzenesulfonyl]-piperidine (Compound 64)

65. (2R, 4R)-4-Acetylamino-2-hydroxyaminocarbonyl-1-[4-(4-hydroxyphenyl) benzenesulfonyl]-piperidine (Compound 65)

66. (2R, 4R)-4-Acetylamino-2-hydroxyaminocarbonyl-1-(4-methoxybenzenesulfonyl)-piperidine (Compound 66)

67. (2R, 4R)-2-Hydroxyaminocarbonyl-4-(4-methylpentanoylamino)-1-[4-(4-nitrophenyl) benzenesulfonyl]-piperidine (Compound 67)

68. (2R, 4R)-2-Carboxy-1-(4-methoxybenzenesulfonyl)-4-(pyridyl -2-yl) carbonylamino-piperidine (Compound 68)

69. (2R, 4R)-2-Hydroxyaminocarbonyl-1-(4-methoxybenzenesulfonyl)-4-(pyridyl -2-yl) carbonylamino-piperidine (Compound 69)

70. (2R, 4R)-2-Hydroxyaminocarbonyl-4-(4-methylpentylamino)-1-(2-thiophenesulfonyl)-piperidine (Compound 70)

71. (2R, 4R)-1-[4-(4-Chlorohenyl)benzenesulfonyl]-2-hydroxyaminocarbonyl-4-(4-methylpentanoylamino)-piperidine (Compound 71)

72. (2R, 4R)-4-Acetylamino-1-[4-(4-chlorohenyl)benzenesulfonyl]-2-hydroxyaminocarbonyl-piperidine (Compound 72)

73. (2R, 4R)-2-Hydroxyaminocarbonyl-1-[4-(4-methoxyphenyl)benzenesulfonyl]-4-(2-pyridinecarbonylamino-piperidine (Compound 73)

74. (2R, 4R)-1-[4-(4-Chlorophenyl)benzenesulfonyl]-2-hydroxyaminocarbonyl-4-(2-pyridinecarbonylamino)-piperidine (Compound 74)

75. (2R, 4R)-2-Hydroxyaminocarbonyl-1-[4-(4-methoxyphenyl)benzenesulfonyl]-4-(4-nitrobenzoylamino)-piperidine (Compound 75)

76. (2R, 4R)-2-Hydroxyaminocarbonyl-4-(3-indolecarbonylamino)-1-[4-(4-methoxyphenyl) benzenesulfonyl]-piperidine (Compound 76)

77. (2R, 4R)-2-Hydroxyaminocarbonyl-4-(4-methylpentanoylamino)-1-(4-nitrobenzenesulfonyl)-piperidine (Compound 77)

78. (2S, 4S)-2-Hydroxyaminocarbonyl-1-(4-methoxybenzenesulfonyl)-4-[5-(4-methoxyphenoxy) pentanoylamino]-piperidine (Compound 78)

79. (2S, 4S)-2-Hydroxyaminocarbonyl-1-(4-methoxybenzenesulfonyl)-4-(4-methylpentanoylamino)-piperidine (Compound 79)

80. (2S, 4S)-2-Hydroxyaminocarbonyl-1-(4-methoxybenzenesulfonyl)-4-(4-methoxybenzoylamino)-piperidine (Compound 80)

81. (2S, 4S)-4-Acetylamino-2-hydroxyaminocarbonyl-1-(4-methoxybenzenesulfonyl)-piperidine (Compound 81)

82. (2R, 4S)-4-Acetylamino-2-hydroxyaminocarbonyl-1-[4-(4-methoxyphenyl) benzenesulfonyl]-piperidine (Compound 82)

83. (2R, 4R)-4-Acetylamino-1-[4-(4- chlorophenyl)benzenesulfonyl]2-(N′-formyl-hydroxyamino) methyl-piperidine (Compound 83)

84. (2R, 4R)-1-[4-(4-Chlorophenyl)benzenesulfonyl]-2-(N′-formyl -hydroxyamino) methyl-4-(4-methylpentanoylamino)-piperidine (Compound 84)

85. (2R, 4R)-2-Carboxy-1-(4-methoxybenzenesulfonyl)-4-(pyridyl-N-oxide-2-yl) carbonylamino-piperidine (Compound 85)

86. (2R, 4R)-2-Carboxy-1-(4-chlorobenzenesulfonyl)-4-(pyridyl-N- oxide-2-yl) carbonylamino-piperidine (Compound 86)

87. (2R, 4R)-2-Carboxy-1-methanesulfonyl-4-(pyridyl-N-oxide-2-yl) carbonylamino-piperidine (Compound 87)

88. (2R, 4R)-2-Carboxy-1-dimethylaminosulfonyl-4-(pyridyl-N-oxide-2-yl) carbonylamino-piperidine (Compound 88)

89. (2R, 4R)-2-Carboxy-1-[4-(4-chlorophenyl)benzenesulfonyl]-4-(pyridyl-N-oxide -2-yl) carbonylamino-piperidine (Compound 89)

The carboxylic acid derivatives in this invention is useful as pharmaceutical compositions using to treatment and/or prevention for disease related to destruction of extra cellular matrix induced by MMPs. They can be administered orally in the form of tablets, capsules, granules and syrups, and also can be administered intravenously. An effective dosage of the compounds is from 10 to 1000 mg once to several times a day for adults, though it may be adjusted depending on age and symptoms.

### PHARMACOLOGICAL EXAMINATION

The compound of general formula (I) in the invention is a potent inhibitor of MMPs. A pharmacological examination is described as follows.

Examination 1: MMP-1 (type I collagenase) inhibitory activities.

Inhibitory activities of MMP-1 (type I collagenase) activities were estimated by enzyme assay using human fibroblasts derived MMP-1 (Yagai Co. Ltd.) as enzyme, and MOCAc-Pro-Leu-Gly-Leu-A₂pr(Dnp)-Ala-Arg-NH₂ (Peptide Research Center, 3163-v) as substrate. Thus, MMP-1 (0.01 U/ml), test compound (10⁻¹⁰˜10⁻⁵ M), and the substrate (10 μM) were incubated in 50 mM Tris-HCl buffer (pH 7.4) containing 0.2M NaCl, 10 mM CaCl₂, 0.02% NaN₃, and 0.05% Brij 35 at 37° C. for 4 h. The reaction was stopped by adding of 4 volumes of sodium acetate buffer (pH 4.0), and the degradation of substrate was calculated by measurement with a fluorescence intensity (λ ex 328 nm, λ em 393 nm). Inhibition of degradation of substrate in the presence of compound was determined by comparison with the degradation percent of substrate in the absence of compound, and concentration of 50% inhibition (IC₅₀ value) was calculated.

Examination 2: MMP-2 (gelatinase A) and MMP-9 (gelatinase B) inhibitory activities.

Pro MMP-2 was obtained from culture medium separated from human pro-MMP-2 cDNA tranfected COS-1 cells. Pro MMP-2 activated by 1 mM (4-aminophenyl) mercury acetic acid and MMP-9, derived human fibrosarcome (Yagai Co. Ltd.), were used as enzyme respectively. Inhibition of both enzyme activities were estimated by enzyme assay using MOCAc-Pro-Leu-Gly-Leu-A₂pr(Dnp)-Ala-Arg-NH₂ (Peptide Research Center, 3163-v) as substrate. Thus, MMP-2 or MMP-9 (0.01 U/ml), test compound (several concentrations), and the substrate (10 μM) were incubated in 50 mM Tris - HCl buffer (pH 7.4) containing 0.2M NaCl, 10 mM CaCl₂, 0.02% NaN₃, and 0.05% Brij 35 at 42° C. for 2 h (MMP-2), 3 h (MMP-9) respectively. Inhibition of degradation of substrate in the presence of compound was determined by comparison with the degradation of substrate in the absence of compound, and concentration of 50% inhibition (IC₅₀ value) was calculated.

Examination 3: Δ MT1-MMP (Δ MMP-14) inhibitory activities.

ProMT1-MMP cDNA encoded extracellular domain of pro Δ MT1-MMP was tranfected E. coli BL21DE3, pro Δ MT1-MMP collected as inclusion body was solved using 8M urea, and it was pulified by ion exchenge column chlomatography. Inhibition of enzyme activities were estimated by enzyme assay using activated Δ MT1-MMP, obtained from activation of pro Δ MT1-MMP by 10 μ/ml tripsine, and MOCAc-Pro-Leu-Gly-Leu-A₂pr(Dnp)-Ala-Arg-NH₂ (Peptide Research Center, 3163-v) as substrate. Thus, Δ MT1-MMP, test compound (several concentrations), and the substrate (10 μM) were incubated in 50 mM Tris - HCl buffer (pH 7.4) containing 0.2M NaCl, 10 mM CaCl₂, 0.02% NaN₃, 0.05% Brij 35, and 10 μM leuipeputine at 37° C. for 30 min. The reaction was stopped by adding of 4 volumes of sodium acetate buffer (pH 4.0), and the degradation of substrate was calculated by measurement with a fluorescence intensity (λ ex 328 nm, λ em 393 nm). Inhibition of degradation of substrate in the presence of compound was determined by comparison with the degradation of substrate in the absence of compound, and concentration of 50% inhibition (IC₅₀ value) was calculated.

The results are described in table 1, 2.

TABLE 1

| Chem. | | | | |
|---|---|---|---|---|
| No. | MMP-1 | MMP-2 | MMP-9 | ΔMMP-14 |
| 45 | 45.3% (10⁻⁵M) | 1.7 × 10⁻⁹M | 1.4 × 10⁻⁹M | 1.6 × 10⁻⁸M |
| 46 | 7.6 × 10⁻⁶M | 1.5 × 10⁻⁹M | 8.8 × 10⁻¹⁰M | 4.3 × 10⁻⁹M |
| 47 | 5.5 × 10⁻⁶M | 1.7 × 10⁻⁹M | 1.3 × 10⁻⁹M | 3.2 × 10⁻⁹M |
| 48 | 35.5% (10⁻⁵M) | 5.5 × 10⁻¹⁰M | 4.0 × 10⁻¹⁰M | 3.1 × 10⁻⁷M |
| 49 | 2.6 × 10⁻⁶M | 2.5 × 10⁻¹⁰M | 2.6 × 10⁻¹⁰M | 1.3 × 10⁻⁷M |
| 50 | 8.4 × 10⁻⁶M | 7.3 × 10⁻⁸M | 4.4 × 10⁻⁸M | 3.5 × 10⁻⁸M |
| 51 | 6.5 × 10⁻⁹M | 8.0 × 10⁻⁸M | 7.5 × 10⁻⁸M | 3.1 × 10⁻⁸M |
| 52 | 48.9% (10⁻⁵M) | 2.1 × 10⁻⁷M | 4.2 × 10⁻⁷M | 7.9 × 10⁻⁸M |
| 53 | 53.0% (10⁻⁵M) | 9.6 × 10⁻⁷M | 6.9 × 10⁻⁶M | 2.0 × 10⁻⁶M |
| 54 | 45.8% (10⁻⁵M) | 3.8 × 10⁻⁷M | 1.8 × 10⁻⁶M | 2.0 × 10⁻⁷M |
| 55 | 29.5% (10⁻⁵M) | 2.4 × 10⁻⁶M | 34.8% (10⁻⁵M) | 3.0 × 10⁻⁶M |
| 56 | 1.3 × 10⁻⁶M | 4.9 × 10⁻¹⁰M | 3.6 × 10⁻¹⁰M | 1.6 × 10⁻⁸M |
| 57 | 4.6 × 10⁻⁷M | 1.8 × 10⁻⁹M | 1.1 × 10⁻⁸M | 1.0 × 10⁻⁷M |
| 59 | 57.6% (10⁻⁵M) | 1.8 × 10⁻⁷M | 1.1 × 10⁻⁸M | 9.5 × 10⁻⁸M |
| 60 | 4.9 × 10⁻⁶M | 2.9 × 10⁻⁹M | 8.9 × 10⁻⁷M | 1.3 × 10⁻⁷M |
| 61 | 44.6% (10⁻⁵M) | 2.7 × 10⁻⁹M | 1.9 × 10⁻⁸M | 4.9 × 10⁻⁸M |
| 62 | 8.7 × 10⁻⁷M | 9.3 × 10⁻¹⁰M | 3.2 × 10⁻⁹M | 4.5 × 10⁻⁸M |
| 63 | 5.1 × 10⁻⁷M | 2.8 × 10⁻⁸M | 3.1 × 10⁻⁸M | 1.7 × 10⁻⁸M |
| 64 | 6.5 × 10⁻⁷M | 9.1 × 10⁻¹⁰M | 7.2 × 10⁻¹⁰M | 8.9 × 10⁻⁹M |
| 65 | 8.7 × 10⁻⁷M | 4.8 × 10⁻⁹M | 5.1 × 10⁻⁸M | 2.3 × 10⁻⁸M |
| 66 | 5.3 × 10⁻⁷M | 4.2 × 10⁻⁹M | 8.6 × 10⁻⁹M | 2.1 × 10⁻⁹M |
| 67 | 1.2 × 10⁻⁶M | 2.2 × 10⁻⁸M | 3.6 × 10⁻⁸M | 1.5 × 10⁻⁶M |
| 69 | 4.1 × 10⁻⁸M | 2.5 × 10⁻⁹M | 1.4 × 10⁻⁹M | 2.4 × 10⁻⁸M |
| 70 | 5.6 × 10⁻⁶M | 7.4 × 10⁻⁷M | 8.3 × 10⁻⁸M | 3.8 × 10⁻⁷M |
| 71 | 6.4 × 10⁻⁷M | 1.9 × 10⁻⁸M | 3.4 × 10⁻⁹M | 2.9 × 10⁻⁷M |
| 72 | 3.7 × 10⁻⁸M | 3.0 × 10⁻⁹M | 1.1 × 10⁻⁹M | 2.3 × 10⁻⁸M |
| 73 | 1.2 × 10⁻⁶M | 6.0 × 10⁻⁹M | 4.0 × 10⁻⁹M | 3.6 × 10⁻⁹M |
| 74 | 48.9% (10⁻⁵M) | 5.2 × 10⁻⁹M | 1.3 × 10⁻⁸M | 7.4 × 10⁻⁸M |
| 75 | 2.5 × 10⁻⁶M | 1.5 × 10⁻⁸M | 1.6 × 10⁻⁸M | 8.9 × 10⁻⁷M |
| 76 | 12.9% (10⁻⁵M) | 1.2 × 10⁻⁸M | 2.1 × 10⁻⁸M | 9.6 × 10⁻⁸M |
| 77 | 32.2% (10⁻⁵M) | 4.8 × 10⁻⁷M | 7.4 × 10⁻⁷M | 2.3 × 10⁻⁶M |
| 78 | 31.1% (10⁻⁵M) | 5.0 × 10⁻⁷M | 4.5 × 10⁻⁷M | 3.0 × 10⁻⁷M |
| 79 | NE (10⁻⁵M) | 7.6 × 10⁻⁶M | 23.3% (10⁻⁵M) | 5.0 × 10⁻⁶M |
| 81 | 37.3% (10⁻⁵M) | 2.2 × 10⁻⁷M | 3.9 × 10⁻⁷M | 8.9 × 10⁻⁸M |
| 82 | NE (10⁻⁵M) | 7.4 × 10⁻⁷M | 4.4 × 10⁻⁷M | 1.3 × 10⁻⁶M |
| 89 | 14.0% (10⁻⁵M) | 2.8 × 10⁻⁸M | 2.0 × 10⁻⁷M | 7.0 × 10⁻⁶M |

IC₅₀ or inhibition percent is described.
NE means no effect.

TABLE 2

| Chem. | | | | |
|---|---|---|---|---|
| No. | MMP-1 | MMP-2 | MMP-9 | ΔMMP-14 |
| 45 | 45.3% (10⁻⁵M) | 1.7 × 10⁻⁹M | 1.4 × 10⁻⁹M | 1.6 × 10⁻⁸M |
| 46 | 7.6 × 10⁻⁶M | 1.5 × 10⁻⁹M | 8.8 × 10⁻¹⁰M | 4.3 × 10⁻⁹M |
| 47 | 5.5 × 10⁻⁶M | 1.7 × 10⁻⁹M | 1.3 × 10⁻⁹M | 3.2 × 10⁻⁹M |
| 48 | 35.5% (10⁻⁵M) | 5.5 × 10⁻¹⁰M | 4.0 × 10⁻¹⁰M | 3.1 × 10⁻⁷M |
| 49 | 2.6 × 10⁻⁶M | 2.5 × 10⁻¹⁰M | 2.6 × 10⁻¹⁰M | 1.3 × 10⁻⁷M |
| 50 | 8.4 × 10⁻⁶M | 7.3 × 10⁻⁸M | 4.4 × 10⁻⁸M | 3.5 × 10⁻⁸M |
| 51 | 6.5 × 10⁻⁹M | 8.0 × 10⁻⁸M | 7.5 × 10⁻⁸M | 3.1 × 10⁻⁸M |
| 52 | 48.9% (10⁻⁵M) | 2.1 × 10⁻⁷M | 4.2 × 10⁻⁷M | 7.9 × 10⁻⁸M |
| 53 | 53.0% (10⁻⁵M) | 9.6 × 10⁻⁷M | 6.9 × 10⁻⁶M | 2.0 × 10⁻⁶M |
| 54 | 45.8% (10⁻⁵M) | 3.8 × 10⁻⁷M | 1.8 × 10⁻⁶M | 2.0 × 10⁻⁷M |
| 55 | 29.5% (10⁻⁵M) | 2.4 × 10⁻⁶M | 34.8% (10⁻⁵M) | 3.0 × 10⁻⁶M |
| 56 | 1.3 × 10⁻⁶M | 4.9 × 10⁻¹⁰M | 3.6 × 10⁻¹⁰M | 1.6 × 10⁻⁸M |
| 57 | 4.6 × 10⁻⁷M | 1.8 × 10⁻⁹M | 1.1 × 10⁻⁸M | 1.0 × 10⁻⁷M |
| 59 | 57.6% (10⁻⁵M) | 1.8 × 10⁻⁷M | 1.1 × 10⁻⁸M | 9.5 × 10⁻⁸M |
| 60 | 4.9 × 10⁻⁶M | 2.9 × 10⁻⁹M | 8.9 × 10⁻⁷M | 1.3 × 10⁻⁷M |
| 61 | 44.6% (10⁻⁵M) | 2.7 × 10⁻⁹M | 1.9 × 10⁻⁸M | 4.9 × 10⁻⁸M |
| 62 | 8.7 × 10⁻⁷M | 9.3 × 10⁻¹⁰M | 3.2 × 10⁻⁹M | 4.5 × 10⁻⁸M |
| 63 | 5.1 × 10⁻⁷M | 2.8 × 10⁻⁸M | 3.1 × 10⁻⁸M | 1.7 × 10⁻⁸M |
| 64 | 6.5 × 10⁻⁷M | 9.1 × 10⁻¹⁰M | 7.2 × 10⁻¹⁰M | 8.9 × 10⁻⁹M |
| 65 | 8.7 × 10⁻⁷M | 4.8 × 10⁻⁹M | 5.1 × 10⁻⁸M | 2.3 × 10⁻⁸M |
| 66 | 5.3 × 10⁻⁷M | 4.2 × 10⁻⁹M | 8.6 × 10⁻⁹M | 2.1 × 10⁻⁹M |
| 67 | 1.2 × 10⁻⁶M | 2.2 × 10⁻⁸M | 3.6 × 10⁻⁸M | 1.5 × 10⁻⁶M |
| 69 | 4.1 × 10⁻⁸M | 2.5 × 10⁻⁹M | 1.4 × 10⁻⁹M | 2.4 × 10⁻⁸M |
| 70 | 5.6 × 10⁻⁶M | 7.4 × 10⁻⁷M | 8.3 × 10⁻⁸M | 3.8 × 10⁻⁷M |
| 71 | 6.4 × 10⁻⁷M | 1.9 × 10⁻⁸M | 3.4 × 10⁻⁹M | 2.9 × 10⁻⁷M |
| 72 | 3.7 × 10⁻⁸M | 3.0 × 10⁻⁹M | 1.1 × 10⁻⁹M | 2.3 × 10⁻⁸M |
| 73 | 1.2 × 10⁻⁶M | 6.0 × 10⁻⁹M | 4.0 × 10⁻⁹M | 3.6 × 10⁻⁹M |
| 74 | 48.9% (10⁻⁵M) | 5.2 × 10⁻⁹M | 1.3 × 10⁻⁸M | 7.4 × 10⁻⁸M |
| 75 | 2.5 × 10⁻⁶M | 1.5 × 10⁻⁸M | 1.6 × 10⁻⁸M | 8.9 × 10⁻⁷M |
| 76 | 12.9% (10⁻⁵M) | 1.2 × 10⁻⁸M | 2.1 × 10⁻⁸M | 9.6 × 10⁻⁸M |
| 77 | 32.2% (10⁻⁵M) | 4.8 × 10⁻⁷M | 7.4 × 10⁻⁷M | 2.3 × 10⁻⁶M |
| 78 | 31.1% (10⁻⁵M) | 5.0 × 10⁻⁷M | 4.5 × 10⁻⁷M | 3.0 × 10⁻⁷M |
| 79 | NE (10⁻⁵M) | 7.6 × 10⁻⁶M | 23.3% (10⁻⁵M) | 5.0 × 10⁻⁶M |
| 81 | 37.3% (10⁻⁵M) | 2.2 × 10⁻⁷M | 3.9 × 10⁻⁷M | 8.9 × 10⁻⁸M |
| 82 | NE (10⁻⁵M) | 7.4 × 10⁻⁷M | 4.4 × 10⁻⁷M | 1.3 × 10⁻⁶M |
| 89 | 14.0% (10⁻⁵M) | 2.8 × 10⁻⁸M | 2.0 × 10⁻⁷M | 7.0 × 10⁻⁶M |

IC₅₀ or inhibition percent is described.
NE means no effect.

### EXAMPLE

The following examples are provided only for the purpose of the compound and not restrict the disclosed invention.

Example: 1 (2R,4R)-2-Carboxy-1-[4-(4-methoxyphenyl)benzenesulfonyl]-4-(4-methylpentanoylamino)-piperidine (Compound 5)

(a) (2R,4S)-4-Hydroxy-2-methoxycarbonyl-1-[4-(4-methoxyphenyl)benzenesulfonyl]-piperidine

(2R,4S)-4-Hydroxypipecolinic acid methyl ester (2.8 g) was dissolved in THF/H₂O (2:1, 45 mL), then NaHCO₃ (1.63 g) and 4-(4-methoxyphenyl)benzenesulfonylchloride (5.47 g) were added at 0° C., and the mixture was stirred for 3 h at room temperature.

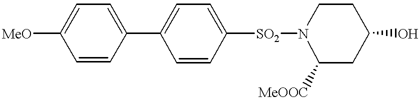

The reaction mixture was concentrated in vacuo, then the residue was extracted with ethyl acetate (100 mL×2) followed by washed with brine, dried (Na₂SO₄), filtered, and concentrated. The residue was purified by silica gel column chromatography using AcOEt:n-hexane (2:1), the object compound was obtained as a white solid (5.45 g).

IR(cm⁻¹): 3514,1746,1608,1341,1296,1197,1152,1080

MS(m/z): 405(M⁺),373,329,247,183,139,82,55(BP)

¹H-NMR(CDCl₃) 1.74-1.77(2H,m,C3-H,C5-H),1.97-2.03(1H,m,C5-H),2.43(1H,brd,C3-H), 3.54(3H,s,CO₂CH₃),3.57-3.61(1H,m,C6-H),3.86(3H,s,OCH₃),4.15(1H,brs,C4-H),4.74(1H, d,C2-H),7.00(2H,d,aromatic),7.56(2H,d,aromatic),7.66(2H,d,aromatic),7.84(2H,d,aromatic)

(b) (2R,4S)-4-Mesyloxy-2-methoxycarbonyl-1-[4-(4-methoxyphenyl)benzenesulfonyl]-piperidine

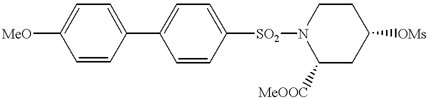

The compound (760 mg) which was prepared by example 1 (a) was dissolved in pyridine (6 mL), then DMAP (50 mg) and mesylchloride (279 mg) were slowly added at 0° C., followed by stirred for 3 h at room temperature. The reaction mixture was poured into 10% aq. HCl and acidified, then extracted with ethyl acetate (40 mL×2) followed by washed with brine, dried (Na₂SO₄), filtered, and concentrated. The resulting solid was filtered, the object compound was obtained as a white solid (0.84 g).

IR(cm⁻¹): 2938,1734,1608,1350,1248,1161

MS(m/z): 483(M⁺),424,387,328,183(BP),140

¹H-NMR(CDCl₃): 1.90-1.94(1H,m,C5-H),2.03(1H,brd,C5-H),2.09-2.16(1H,m,C3-H), 2.71(1H,brd,C3-H),2.93(3H,s,Ms),3.54-3.61(1H,m,C6-H),3.58(3H,s,CO₂CH₃), 3.81(1H, dd,C6-H),3.87(3H,s,OCH₃),4.85(1H,d,C2-H),5.05(1H,brS,C4-H), 7.01 (2H,d,aromatic),7.56(2H,d,aromatic),7.67(2H,d,aromatic),7.84(2H,d,aromatic)

(c) (2R,4R)-4-Azide-2-methoxycarbonyl-1-[4-(4-methoxyphenyl)benzenesulfonyl]-piperidine

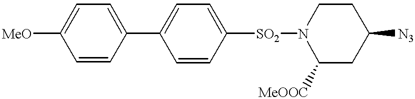

The compound (3 g) which was prepared by example 1 (b) was dissolved in DMF (20 mL), then sodium azide (605 mg) was added and stirred on overnight at 80° C. The reaction mixture was poured into ice water, then extracted with ethyl acetate (100 mL) followed by washed with brine, dried (Na₂SO₄), filtered, and concentrated. The residue was purified by silica gel column chromatography using AcOEt:n-hexane (1:3), the object compound was obtained as a white solid (2.67 g).

IR(cm⁻¹): 2944,1740,1608,1521,1341,1290,1158

MS(m/z): 430(M⁺),402,343,183(BP), 139

¹H-NMR(CDCl₃): 1.49-1.58(1H,m,C5-H),1.74(1H,dt,C3-H),1.97(1H,brd,C5-H), 2.38(1H,brd,C3-H),3.29(1H,dt,C6-H),3.37-3.49(1H,m,C4-H),3.57(3H,s,CO₂CH₃), 3.87(3H,s,OCH₃),3.94(1H,brd,C6-H),4.92(1H,d,C2-H),7.01 (2H,d,aromatic), 7.56(2H,d,aromatic),7.67(2H,d,aromatic),7.82(2H,d,aromatic)

(d) (2R,4R)-4-Amino-2-methoxycarbonyl-1-[4-(4-methoxyphenyl)benzenesulfonyl]-piperidine

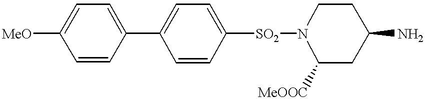

The compound (4.74 g) which was prepared by example 1 (c) was dissolved in MeOH/THF (3:2), 50 mL), then 5% Pd-C (500 mg) was added and stirred for 24 h under H₂ atmosphere. The reaction mixture was filtered on celite pad, and concentrated. The residue was purified by silica gel column chromatography using CHCl₃:MeOH (20:1), the object compound was obtained as a pale yellow oil (4.33 g).

IR(cm⁻¹): 2938,1737,1290,1158,1038

MS(m/z): 404(M⁺),345,247,183,114,56(BP)

¹H-NMR(CDCl₃): 1.29-1.35(1H,m,C5-H),1.53(1H,dt,C3-H),1.81 (1H,brd,C3-H), 2.26-2.31(1H,m,C3-H),2.70-2.77(1H,m,C4-H),3.29(1H,dt,C6-H),3.55(3H,s,CO₂CH₃), 3.86(3H,s,OCH₃),3.86-3.91(1H,m,C6-H),4.88(1H,d,C2-H),7.00(2H,d,aromatic), 7.55(2H,d,aromatic),7.66(2H,d,aromatic),7.82(2H,d,aromatic)

(e) (2R,4R)-4-(4-methylpentanoyl)amino-2-methoxycarbonyl-1-[4-(4-methoxyphenyl) benzenesulfonyl]-piperidine

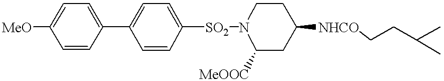

The compound (500 mg) which was prepared by example 1 (d) and isocaproic acid (0.19 mL) were dissolved in DMF/CH₂Cl₂ (5:1, 6 mL), then WSCDI (284 mg), N-methylmorpholine (0.16 mL), and DMAP (10 mg) were added at 0° C. and stirred on overnight. The reaction mixture was poured into water, then extracted with ethyl acetate (50 mL) followed by washed with brine, dried (Na₂SO₄), filtered, and concentrated. The resulting solid was filtered, the object compound was obtained as a white solid (500 mg).

¹H-NMR(CDCl₃): 0.89(6H,d,CH₃), 1.38-1.67(5H,m,CH₂,CH,C5-H),2.00(1H,d,C3-H), 2.13(2H,m,CH₂)2.42(1H,d,C3-H),3.35 (1H,dt,C6H),3.55 (3H,s,CO₂CH₃),3.87(3H,s,OCH₃ ), 3.85-3.91 (2H,m,C4-H,C6-H),4.91 (1H,d,C2-H),5.24(1H,d,NH),7.01 (2H,d,aromatic), 7.56(2H,d,aromatic),7.66(2H,d,aromatic),7.82(2H,d,aromatic)

(f) (2R,4R)-2—Carboxy4-(4-methylpentanoyl)amino-1-[4-(4-methoxyphenyl)benzene sulfonyl]-piperidine

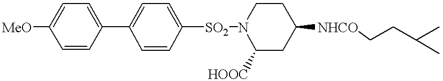

The compound (181 mg) which was prepared by example 1 (e) was dissolved in THF/H₂O (3:1, 4 mL), then lithium hydroxide monohydrate (30 mg) was added and stirred on overnight. After the reaction mixture was neutralized at 10% aq.HCl, it was extracted with chloroform (50 mL×2) followed by washed with brine, dried (Na₂SO₄), filtered, and concentrated. The residue was purified by silica gel column chromatography using CHCl₃:MeOH (10:1), the object compound was obtained as a white solid (175 mg).

IR(cm⁻¹): 3400,2944,1737,1608,1335,1251,1152,819

MS(m/z): 470(M⁺-18),236,149,111,83,57(BP)

¹H-NMR(CDCl₃): 0.86(6H,d,CH₃),1.24-1.62 (5H,m,CH₂,CH,C5-H) ,1.78(1H,d,C3-H), 2.13(2H,m,CH₂)2.40(1H,d,C3-H),3.21 (1H,m,C6-H),3.66(1H,m,C6-H),3.86(3H,s,OCH₃), 3.92(1H,m,C4-H),4.86(1H,d,C2-H),5.63(1H,d,NH),7.00(2H,d,aromatic),7.54(2H,d,aromatic), 7.60(2H,d,aromatic),7.80(2H,d,aromatic)

Example: 2 (2R,4R)-2-Hydroxyamincarbonyl-1-[4-(4-methoxyphenyl)benzenesulfon-yl]-4-(4-methylpentanoylamino)-piperidine (Compound 49)

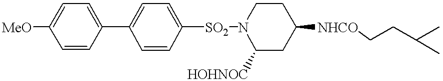

The compound (486 mg) which was prepared by example 1 and HOBT (228 mg) were dissolved in DMF/CH₂Cl₂ (2:1, 3 mL), then WSCDI (286 mg) and N-methylmorpholine (0.33 mL) were added at 0° C. and stirred for 30 min, followed by addition of o-benzylhydroxyamine hydrochloride salt and further stirred on overnight. The reaction mixture was poured into a water and extracted with ethyl acetate (100 mL), followed by washed with brine, dried (Na₂SO₄), filtered, and concentrated.

The resulting solid was filtered, a white solid (590 mg) was obtained. Subsequently, the compound (590 mg) was dissolved in THF (3 mL) then 5% Pd-C was added and stirred on overnight under H₂ atmosphere. The reaction mixture was filtered on celite pad, and concentrated. The residue was purified by silica gel column chromatography using CHCl₃:MeOH (10:1), the object compound was obtained as a white solid (450 mg).

IR(cm⁻¹):3256,2944,1647,1521,1305,1251,1152,1032

MS(m/z):442(M⁺-62),327,248,183,139,59(BP)

¹H-NMR(CDCl₃):0.87(6H,d,CH₃),1.23-1.81 (5H,m,CH₂,CH,C5-H),1.92(1H,d,C3-H), 2.11(2H,t,CH₂)2.33(H,d,C3-H),3.44(H,m,C4-H),3.86(3H,s,OCH₃),3.92(1H,m,C6-H), 4.00(1H,m,C6-H),4.71(1H,m,C2-H),5.49(1H,d,NH),6.99(2H,d,aromatic), 7.56(2H,d,aromatic),7.67(2H,d,aromatic),7.84(2H,d,aromatic),9.92(1H,brs,NH)

Example: 3 (2R,4R)-4-Acetylamino-2-carboxy-1-[4-(4-chlorophenyl)benzenesulfonyl]piperidine (Compound 31)

(a) (2R,4R)-4-Acetylamino-2-methoxycarbonyl-1-(4-bromobenzenesulfonyl)-piperidine

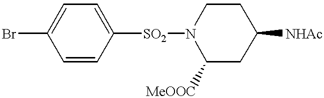

(2R,4R)-4-Azide-2-methoxycarbonyl-1-(4-bromobenzenesulfonyl)-piperidine (1.97 g) was dissolved in THF/H₂O (4:1, 16 mL), then triphenylphosphine (1.29 g) was added and stirred for 3 h at room temperature under Ar atmosphere. Subsequently, sat.Na₂CO₃ (6 mL) was added and stirred for 30 min, then concentrated and extracted with chloroform (50 mL×2), dried (Na₂SO₄), filtered, and concentrated. The crude product was dissolved in dichloromethane (10 mL), then triethylamine (1.02 mL), and acetic anhydride (0.69 mL) were added at 0° C., and stirred for 12 h. The reaction mixture was concentrated, then diluted with ethyl acetate (50 mL) followed by washed with 10% aq.citric acid, sat.NaHCO₃, and brine. Subsequently, it was dried (Na₂SO₄), filtered, and concentrated, then the residue was purified by silica gel column chromatography using AcOEt:n-hexane (1:5), the object compound was obtained as a pale yellow oil (1.60 g).

IR(cm⁻¹):2938,1740,1653,1545,1437,1338,1158,1089

MS(m/z):419(M⁺),361,300(BP),277,221,140,108,80,56

¹H-NMR(CDCl₃): 1.39(1H,m,C5-H),1.64(1H,m,C5-H),1.95(3H,s,Ac),2.00(1H,m,C6-H), 2.42(1H,m,C3-H),3.27(1H,td,C6-H),3.58(3H,s,CO₂CH₃),3.84(2H,m,C4,C6-H), 4.87(1H,d,C2-H),5.40(1H,brd,NH),7.65(4H,m,aromatic)

(b) (2R,4R)-4-Acetylamino-2-carboxy-1-[4-(4-chlorophenyl)benzenesulfonyl)-piperidine (Compound 31)

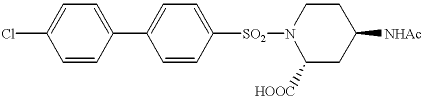

The compound (1.50 g) which was prepared by example 3 (a) was dissolved in acetonitrile (12 mL), then 4-chlorophenylboronic acid (839 mg), cesium fluoride (924 mg), and tetrakis(triphenylphosphine) palladium (207 mg) were added under Ar atmosphere, and stirred on overnight. The reaction mixture was poured into sat.Na₂CO₃, then extracted with ethyl acetate (50 mL×2), followed by dried (Na₂CO₄), filtered, concentrated. The crude product was dissolved in THF/H₂O (4:1, 10 mL), then lithium hydroxide monohydrate was added at room temperature and stirred on overnight. The reaction mixture was concentrated, then the residue was washed with ether and aqueous layer was neutralized at 10% aq.HCl, extracted with chloroform (50 mL×2), dried (Na₂SO₄), filtered and concentrated. The resulting solid was filtered, the object compound was obtained as a white solid (1.27 g).

IR(cm⁻¹):3364,1728,1629,1545,1326,1152,1092,813,609

ESI-MS(m/z): [M⁺H]⁺437

¹H-NMR(CD₃OD): 1.41 (1H,m,C5-H),1.65(1H,td,C5-H), 1.94(4H,m,C3-H,Ac), 2.42(1H,m,C3-H),3.37(1H,m,C4-H),3.85(2H,m,C6-H),4.88(1H,d,C2-H), 7.45 (2H,d,aromatic),7.54(2H,d,aromatic),7.66(2H,d,aromatic),7.88(2H,d,aromatic)

Example: 4 (2R,4R)-4-Acethylamino-1-[4-(4-chlorophenyl)benzenesulfonyl]-2-hydroxyamino carbonyl-piperidine (Compound 72)

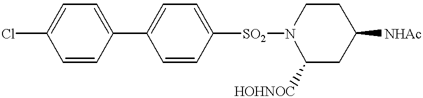

(COCl)₂/CH₂Cl₂ was added to DMF/CH₂Cl₂ at −15° C. and stirred for 30 min. The reaction mixture was concentrated, then the residue was dissolved in CH₂Cl₂ (2 mL) and the compound (145 mg) which was prepared by example 3 was added at 0° C. and stirred for 30 min. The reaction mixture was added other reaction mixture that hydroxylamine hydrochloride salt was dissolved in CH₂Cl₂ then triethylamine was added at 0° C. and stirred for 15 min, and further stirred. After the reaction was completed, poured into H₂O, extracted with chloroform (50 mL×2), dried (Na₂SO₄), filtered, concentrated. The residue was purified by silica gel column chromatography using CHCl₃: MeOH (10:1), the object compound was obtained as a white solid (115 mg).

IR(cm⁻¹):3298,3082,1641,1536,1332,1149,1092

ESI-MS(m/z):[M⁺H]+452

¹H-NMR(DMSO) 1.23(1H,m,C5-H), 1.47(1H,m,C5-H), 1.84(4H,m,C3-H,Ac), 2.14(1H,m,C3-H),3.72(2H,t,C6-H),3.89(1H,m,C6-H),3.99(1H,m,C4-H),4.60(1H,d,C2-H), 7.70(2H,d,aromatic),7.81 (H,d,NH),7.93(2H,d,aromatic),7.96(2H,d,aromatic), 8.02(2H,d,aromatic),9.02(1H,brs,OH),10.8(1H,brs,NHOH)

Example: 5 (2R,4R)-2-Carboxy-1-[4-(4-methoxyphenyl)benzenesulfonyl]-4-(pyridyl-2-yl)carbonyl amino-piperidine (Compound 33)

(a) (2R,4R)-2-Methoxycarbonyl-1-[4-(4-methoxyphenyl)benzenesulfonyl]-4-(pyridyl-2-yl) carbonyl amino-piperidine

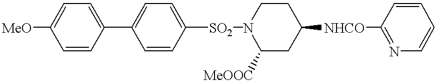

The compound (568 mg) which was prepared by example 1 (d) was dissolved in DMF/CH₂Cl₂ (4:1, 5 mL), then picolinic acid (225 mg), WSCDI (403 mg), N-methylmorpholine (0.23 mL), and DMAP (10 mg) were added at 0° C. and stirred for 3 h. The reaction mixture was poured into H₂O, then extracted with ethyl acetate (50 mL), washed with brine, dried (Na₂SO₄), filtered, concentrated. The residue was purified by silica gel column chromatography using AcOEt:n-hexane (1:5), the object compound was obtained as a white solid (776 mg).

IR(cm⁻¹):3004,1740,1521,1338,1155

¹H-NMR(CDCl₃): 1.60(1H,m,C5-H), 1.83(1H,td,C5-H),2.11 (1H,m,C3-H),2.54(1H,m,C3-H), 3.44(1H,m,C6-H),3.59(3H,s,CO₂CH₃),3.87(3H,s,OCH₃),3.96(1H,m,C6-H), 4.07(1H,m,C4-H),4.97(1H,d,C2-H),7.01 (2H,d,aromatic),7.44(1H,m,NH), 7.57(2H,d,aromatic),7.68(2H,d,aromatic),7.83-7.87(3H,m,aromatic,pyridine), 7.90(1H,d,pyridine),8.16(1H,d,pyridine),8.54(1H,d,pyridine)

(b) (2R,4R)-2-Carbonyl-1-[4-(4-methoxyphenyl)benzenesulfonyl]-4-(2-pyridinecarbonylamino)-piperidine (Compound 33)

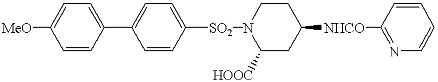

The compound (713 mg) which was prepared by example 5 (a) was dissolved in THF/H₂O (4:1, 5 mL), then lithium hydroxide monohydrate (88 mg) was added at room temperature, and stirred for 3 h. The reaction mixture was neutralized with aq.citric acid, and extracted with chloroform (50 mL×2), dried (Na₂SO₄), filtered concentrated. The resulting solid was filtered, the object compound was obtained as a white solid (693 mg).

IR(cm⁻¹):3352,1734,1530,1358,1156

ESI-MS(m/z):[M⁺H]+496

¹H-NMR(CDCl₃): 1.46(1H,m,C5-H), 1.80(1H,td,C5-H), 1.91 (1H,m,C3-H),2.54(1H,m,C3-H), 3.33(1H,m,C6-H),3.76(1H,m,C6-H),3.86(3H,s,OCH₃),4.18(1H,m,C4-H),4.96(1H,d,C2-H), 6.97(2H,d,aromatic),7.45(1H,dd,NH),7.53(2H,d,aromatic),7.62(2H,d,aromatic),7.81-7.85 (3H,m,aromatic,pyridine),8.09(1H,d,pyridine),8.13(1H,d,pyridine),8.54(1H,d,pyridine)

Example: 6 (2R,4R)-2-Hydroxyaminocarbonyl-1-[4-(4-methoxyphenyl)benzenesulfonyl]-4-(pyridyl-2-yl) carbonylamino-piperidine (Compound 73)

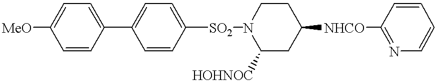

The compound (250 mg) which was prepared by example 5 (compound 33) and HOBT (116 mg) were dissolved in DMF/CH₂Cl₂ (4:1, 5 mL), then WSCDI (145 mg), N-methylmorpholine (0.17 mL), and o-benzylhydroxylamine hydrochloride salt (121 mg) were added at 0° C., and stirred on overnight. The reaction mixture was poured into H₂O, then extracted with ethyl acetate (100 mL), washed with brine, dried (Na₂SO₄), filtered, concentrated. The resulting solid was filtered, and dissolved in THF (5 mL). Subsequently, 5% Pd-C was added and stirred for 6 h at 40° C. under H₂ atmosphere. The reaction mixture was filtered on celite pad, and concentrated. The residue was purified by silica gel column chromatography using CHCl₃:MeOH (50:1), the object compound was obtained as a white solid (237 mg).

IR(cm⁻¹):3346,1665,1521,1335,1155,1035,753

ESI-MS(m/z):[M⁺H]+511

¹H-NMR(CDCl₃): 1.53(1H,m,C5-H),1.69(1H,m,C5-H),2.06(1l H,m,C3-H),2.43(1H,m,C3-H), 3.61 (1H,t,C6-H),3.86(3H,s,OCH₃),4.00(1H,m,C6-H),4.35(1H,m,C4-H),4.78(1H,d,C2-H), 7.00(2H,d,aromatic),7.43(1H,m,NH),7.57(2H,d,aromatic),7.69(2H,d,aromatic),7.83-7.93 (5H,m,aromatic,pyridine),8.21(1H,d,NH),8.50(1H,d,pyridine),10.3(1H,s,OH)

Example: 7 (2R,4R)-4-Benzylamino-2-carboxy-1-[4-(4-methoxyphenyl)benzene-sulfonyl]-piperidine (Compound 14)

(a) (2R,4R)-4-Benzylamino-2-methylcarbonyl-1-[4-(4-methoxyphenyl)benzenesulfonyl]-piperidine

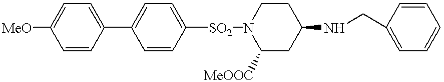

The compound (850 mg) which was prepared by example 1 (d) was dissolved in MeOH (5 mL), then benzaldehyde (1.11 g) was added at room temperature, and stirred for 30 min. After cooled at 0° C., NaBH₄ (224 mg) was added and stirred for 10 min, following stirred for 10 min at room temperature. The reaction mixture was poured into brine, then extracted with ethyl acetate (10 mL×2), dried (Na₂SO₄), filtered, concentrated. The residue was purified by silica gel column chromatography using ethyl acetate:n-hexan (2:1), the object compound was obtained as a white solid (535 mg).

IR(cm⁻¹):1743,1338,1254,1158,1095,753,624

MS(m/z):494(M⁺),435,315,247,183,146,91 (BP)

¹H-NMR(CDCl₃): 1.31-1.38(1H,m,C5-H),1.55(1H,dt,C3-H),1.88(1H,brd,C5-H), 2.69(1H,brd,C3-H),2.54-2.60(1H,m,C4-H),3.26(1H,dt,C6-H),3.54(3H,s,CO₂CH₃), 3.77(2H,s,benzyl positopn),3.87(3H,s,OCH₃),3.90(1H,brd,C6-H),4.89(1H,d,C2-H), 7.00(2H,d,aromatic),7.21-7.34(5H,m,aromatic),7.55(2H,d,aromatic), 7.65(2H,d,aromatic),7.82(2H,d,aromatic)

(b) (2R,4R)-4-Benzylamino-2-carboxy-1-[4-(4-methoxyphenyl)benzenesulfonyl]-piperidine (Compound 14)

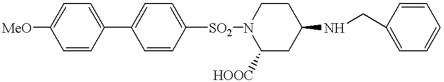

The compound (100 mg) which was prepared by example 7 (a) was dissolved in MeOH/THF (2:1), 1.5 mL), then 1 N aq.LiOH (0.4 mL) was added at 0° C., and stirred for 2 h at room temperature. The reaction mixture was neutralized at 10% aq.HCl, then resulting solid was filtered, the object compound was obtained as a white solid (68 mg).

IR(cm⁻¹):3424,1608,1335,1293,1149

MS(m/z):462(M⁺-18),371,248,215,183,139,91 (BP),

¹H-NMR(DMSO): 1.16-1.22(1H,m,C5-H), 1.44-1.52(1H,m,C3-H),2.02(1H,brd,C5-H), 2.38(1H,brd,C3-H),2.84-2.88(1H,m,C4-H),3.42-3.53(1H,m,C6-H),3.73-3.75 (2H,m,benzyl positopn),3.80(3H,s,OCH₃),3.97-4.02(1H,m,C6-H),4.48(1H,brs,C2-H), 7.02(2H,d,aromatic),7.27-7.43(5H,m,aromatic),7.58(2H,d,aromatic), 7.69(2H,d,aromatic),7.78(2H,d,aromatic)

Example: 8 (2R,4R)-4-Benzylamino-2-hydroxyaminocarbonyl 1-[4-(4-methoxyphenyl) benzenesulfonyl]-piperidine (Compound 56)

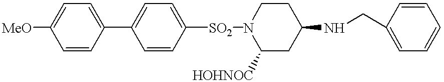

Hydroxylamine hydrochloride salt (200 mg) was dissolved in MeOH (2 mL), then potassium hydroxide (250 mg)/MeOH (1.3 mL) was added at 0° C., and stirred for 1 h. After filtered insoluble material, the filterate was added to the solution that the compound (200 mg) which was prepared by example 7 (a) was dissolved in THF (2 mL), at 0° C., and stirred for 6 h at room temperature. The reaction mixture was neutralized with aq.HCl, then extracted with THF (10 mL×2), dried (Na₂SO₄), filtered, concentrated. The residue was purified by silica gel column chromatography using CHCl₃: MeOH (10:1), the object compound was obtained as a white solid (51 mg).

IR(cm⁻¹):3370,1668,1485,1335,1251,1155,1035

MS(m/z):494(M⁺-1),463,435,371,315,247,183,139,91 (BP)

¹H-NMR(CD₃OD): 1.51-1.56(1H,m,C5-H), 1.71(1H,dt,C3-H),2.22(1H,brd,C5-H), 2.49(1H,dd,C3-H),3.59(1H,dt,C4-H),3.68-3.74(1H,m,C6-H),3.89(3H,s,OCH₃), 4.11 (1H,brd,C6-H),4.22(2H,s,benzyl positopn),4.80(1H,d,C2-H),7.09(2H,d,aromatic), 7.46-7.50(5H,m,aromatic),7.70(2H,d,aromatic),7.86(2H,d,aromatic),7.94(2H,d,aromatic)

Example: 9 (2R,4R)-2-Carboxy4-(4-methylpentanoylamino)-1-(2-thiophenesulfonyl)-piperidine (Compound 7)

(a) (2R,4R)-2-Methoxycarbonyl-4-(4-methylpentanoylamino)-1-(2-thiophenesulfonyl)-piperidine

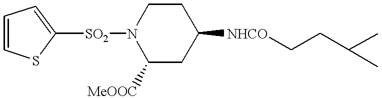

(2R,4R)-2-Methoxycarbonyl-4-(4-methylpentanoylamino)-piperidine (250 mg) was dissolved in CH₂Cl₂ (5 mL), then Et₃N (0.2 mL) and 2-thiophenesulfonyl chloride (267 mg) were added at 0° C., and stirred for 1 h. The reaction mixture was poured into aq.citric acid, then extracted with ethyl acetate (50 mL), washed with brine, dried (Na₂SO₄), filtered, concentrated. The residue was purified by silica gel column chromatography using ethyl acetate:n-hexane (1:10), the object compound was obtained as a white solid (191 mg).

IR(cm⁻¹):3256,1740,1638,1554,1455,1338,1227,1152

MS(m/z):401(M⁺),255,228, 140(BP), 116,80,55

¹H-NMR(CDCl₃):0.89(6H,d,CH₃), 1.37-1.67(5H,m,CH₂,CH,C5-H),2.02(1H,d,C3-H), 2.13(2H,t,CH₂)2.42(1H,dd,C3-H),3.39(1H,dt,C4-H),3.602and3.604(total3H,eachs,CO₂CH₃), 3.84-3.93(2H,m,C4-H,C6-H),4.89(1H,d,C2-H),5.25(1H,d,NH),7.09(1H,m,thiophene), 7.55-7.59(2H,m,thiophene)

(b) (2R,4R)-2-Carboxy-4-(4-methylpentanoylamino)-1-(2-thiophenesulfonyl)-piperidine (Compound 7)

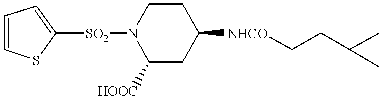

The compound (536 mg) which was prepared by example 9 (a) was dissolved in THF/H₂O (2:1, 6 mL), then lithium hydroxide monohydrate (88 mg) was added, and stirred for 2 h. The reaction mixture was neutralized with aq.citric acid, then extracted with chloroform (50 mL×2), dried (Na₂SO₄), filtered, concentrated. The residue was purified by silica gel column chromatography using CHCl₃:MeOH (5:1), the object compound was obtained as a white solid (357 mg).

IR(cm⁻¹):3364,1737,1626,1545,1338,1149

MS(m/z):387(M⁺),27 1,228,126(BP),82,55

¹H-NMR(CDCl₃):0.89(6H,d,CH₃), 1.30-1.66(5H,m,CH_{2,}CH,C5-H), 1.90(1H,d,C3-H), 2.16(2H,m,CH₂)2.43(1H,d,C3-H),3.33(1H,m,C4-H),3.77(1H,m,C6-H),3.95(1H,m,C6-H), 4.87(1H,d,C2-H),5.59(1H,d,NH),7.05(1H,dd,thiophene),7.53-7.57(2H,m,thiophene)

Example: 10 (2R,4R)-2-Hydroxyaminocarbonyl-4-(4-methylpentanoylamino)-1-(2-thiophene-sulfonyl)-piperidine (Compound 51)

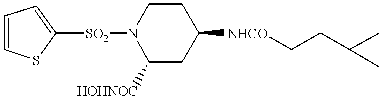

The compound (200 mg) which was prepared by example 9 (compound 7) and HOBT (95 mg) were dissolved in DMF/CH₂Cl₂ (1:1, 4 mL), then WSCDI (119 mg) and N-methylmorpholine (0.07 mL) were added at 0° C., and stirred for 1 h, following addition of hydroxylamine hydrochloride salt (54 mg) and N-methylmorpholine (0.08 mL), and further stirred for 6 h. The reaction mixture was poured into H₂O, then extracted with ethyl acetate (50 mL), washed with brine, dried (Na₂SO₄), filtered, concentrated. The residue was purified by silica gel column chromatography using CHCl₃: MeOH (10:1), the objected compound was obtained as a white solid (40 mg).

IR(cm⁻¹):3280,1644,1539,1341,1152

MS(m/z):338(M⁺-14),358,301,271,228,196,148,116(BP),82,56

¹H-NMR(CDCl₃):0.86(6H,d,CH₃), 1.26-1.53(5H, m,CH₂,CH,C5-H), 1.96(1H,d,C3-H), 2.11 (2H,m,CH₂)2.22(1H,d,C3-H),3.59(1H,m,C4-H),3.90(1H,m,C6-H),4.03(1H,m,C6-H), 4.69(1H,m,C2-H),6.08(1H,m,NH),7.11(1H,m,thiophene),7.60-7.63(2H,m,thiophene), 8.29(1H,brs,NH), 10.4(1H,brs,OH)

Example: 11 (2R,4R)-2-Carboxy-1-[4-(4-methoxyphenyl)benzenesulfonyl]-4-(pyridin-N-oxide-2-yl) carbonylamino-piperidine (Compound 39)

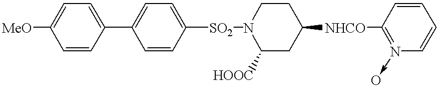

The compound (70 mg) which was prepared by example 5 was dissolved CH₂Cl₂ (5 mL), then mCPBA (73 mg) was added and stirred on overnight at 60° C. The reaction mixture was poured into sat.Na₂S₂O₃, then extracted with CHCl₃ (50 mL), dried (Na₂SO₄), filtered, concentrated. The resulting solid filtered, the object compound was obtained as a white solid (43 mg).

IR(cm⁻¹):3406,1725,1629,1593,1359,1212,1152,1038

TSP-MS(m/z):[M⁺H]+512,[M−H]-510

¹H-NMR(DMSO): 1.38(1H,m,C5-H), 1.69(1H,m,C5-H),2.02(1H,m,C3-H),2.48(1H,m,C3-H), 3.58(1H,m,C4-H),3.88(1H,m,C6-H),3.94(3H,s,OCH₃),4.01 (1H,m,C6-H),4.77(1H,m,C2-H), 7.19(2H,d,aromatic),7.71 (2H,m,pyridine),7.84(2H,d,aromatic),7.95(2H,d,aromatic), 7.99(2H,d,aromatic),8.28(1H,dd,pyridine),8.51(1H,m,pyridine), 11.2(1H,d,NH)

Example: 12 (2R,4R)-4-Acetylamino-2-carboxy-1-(4-methoxybenzenesulfonyl)-piperidine (Compound 24)

(a) (2R,4R)-4-Acetylamino-2-methoxycarbonyl-1-(4-methoxybenzenesulfonyl)-piperidine

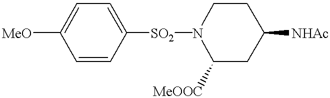

(2R,4R)-4-amino-2-methylcarbonyl-1-(4-methoxybenzenesulfonyl)-piperidine (1.38 g) was dissolved in CH₂Cl₂ (10 mL), then Et₃N (0.88 mL) and acetic anhydride (0.59 mL) were added at 0° C., and stirred for 1 h. The reaction mixture was concentrated, then diluted with ethyl acetate (50 mL), and washed with 10% aq.citric acid, sat.NaHCO₃, and brine, respectively. The organic layer was dried (Na₂SO₄), filtered, concentrated, then the residue was purified by silica gel column chromatography using ethyl acetate:n-hexane (1:5), the object compound was obtained as a colorless oil (1.56 g).

IR(cm⁻¹):3364,1725,1599,1497,1332,1260,1089,555

MS(m/z):338[M⁺-18]252,214,171,140,107,80(BP)55

¹H-NMR(CDCl₃): 1.35(1H,m,C5-H),1.61 (1H,td,J=12.7,5.9 Hz,C5-H),1.86(1H,m,C3-H), 1.96(3H,s,Ac),2.40(1H,m,C3-H),3.23(1H,m,C6-H),3.71 (1H,m,C6-H),3.85(3H,s,Ome), 3.90(1H,m,C4-H),4.83(1H,d,J=5.4 Hz,C2-H),5.86(1H,d,J=8.3 Hz,NH), 6.93(2H,d,J=8.8 Hz,aromatic),7.74(2H,d,J=8.8 Hz,aromatic)

(b) (2R,4R)-4-Acetylamino-2-carboxy-1-(4-methoxybenzenesulfonyl)-piperidine (Compound 24)

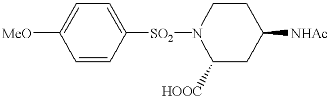

The compound (1.50 g) which was prepared by example 12 (a) was carried out same reaction of example 3 (b), thus the object compound was a white solid (1.30 g).

Example: 13 (2R,4R)-4-Acetylamino-2-hydroxyaminocarbonyl-1-(4-methoxybenzenesulfonyl)-piperidine (Compound 66)

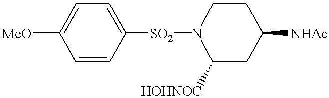

The compound (670 mg) which was prepared by example 12 was carried out same reaction of example 2, thus the object compound was obtained as a colorless solid (265 mg).

IR(cm⁻¹):3262,2926,1659,1545,1497,1260,1149,558

MS(m/z):327[M⁺-44]284,252,171,139,97(BP)77,56

¹H-NMR(DMSO): 1.17(1H,m,C5-H),1.42(1H,m,C5-H),1.80(1H,m,C3-H),1.84(3H,s,Ac), 1.96(1H,m,C3-H),3.66(1H,m,C6-H),3.81(1H,m,C6-H),3.96(4H,s,OmeandC4-H), 4.53(1H,d,J=5.4 Hz,C2-H),7.21 (2H,d,J=8.8 Hz,aromatic),7.79(1H,d,J=8.3 Hz,NH), 7.82(2H,d,J=8.8 Hz,aromatic)8.99(1H,s,CONH),10.8(1H,s,OH)

Example: 14 (2R,4S)-4-Acetylamino-2-carboxy-1-[4-(4-methoxyphenyl)benzenesulfonyl]-piperidine (Compound 44)

(a) (2R,4R)-4-Acetoxy-2-methoxycarbonyl-1-[4-(4-methoxyphenyl)benzenesulfonyl]-piperidine

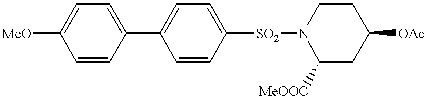

The compound (10.67 g) which was prepared by example 1 (b) was dissolved in toluene (110 mL), then cesium acetate (42.4 g) and 18-crown-6-ether (5.84 g) were added, and reflux for 3 h. The reaction mixture was filtered and concentrated, then the residue was purified by silica gel column chromatography using ethyl acetate:n-hexane (1:4), the object compound was obtained as a colorless solid (3.04 g).

IR(cm⁻¹):2938,2830,1740,1605,1518,1440,1341,1290,1155

MS(m/z):447[M⁺]-373,328,291,247,183,140,79(BP)

¹H-NMR(CDCl₃): 1.53-1.61 (1H,m,C5-H), 1.79-1.86(1H,m,C5-H),2.02(4H,S+m,Ac,C3-H), 2.38-2.42(1H,m,C3-H),3.35(1H,dt,C6-H),3.57(3H,s,CO₂CH₃),3.87(3H,s,OCH₃), 3.89-3.93(1H,m,C6-H),4.73-4.79(1H,m,C4-H),4.92(1H,d,J=6.0 Hz,C2-H), 7.01,7.56(4H,ABq,aromatic)7.67,7.83(4H,ABq,aromatic)

(b) (2R,4R)-4-Hydroxy-2-methoxycarbonyl-1-[4-(4-methoxyphenyl)benzenesulfonyl]-piperidine

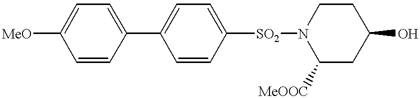

The compound (3.04 g) which was prepared by example 14 (a) was dissolved in MeOH (40 mL), then sodium methoxide (324 mg) was added, and stirred for 1 h at room temperature. Furthermore amberlite IR-120 (2.0 g) was added, and stirred for 1 h. Subsequently, the reaction mixture was filtered and concentrated, then the residue was purified by silica gel column chromatography using ethyl acetate:n-hexane (1:1), the object compound was obtained as a colorless solid (1.85 g).

IR(cm⁻¹):3526,2944,1737,1605,1521,1440,1338,1152

MS(m/z):405[M⁺]373,329,247,183,139,114,82,55(BP)

¹H-NMR(CDCl₃): 1.45-1.53(1H,m,C5-H),1.65-1.72(1H,m,C5-H),1.92-1.96(1H,m,C3-H), 2.38-2.42(1H,m,C3-H),3.29(1H,dt,J=9.2 Hz,C4-H),3.57(3H,s,CO₂CH₃), 3.69-3.71(1H,m,C6-H),3.87(3H,s,OCH₃),3.88-3.93(1H,m,C6-H),4.90(1H,d,J=6.0 Hz,C2-H), 7.00,7.58(4H,ABq,aromatic)7.65,7.84(4H,ABq,aromatic)

(c) (2R,4R)-4-Mesyloxy-2-methoxycarbonyl-1-[4-(4-methoxyphenyl)benzenesulfonyl]-piperidine

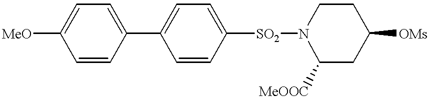

The compound (1.85 g) which was prepared by example 14 (b) was carried out same reaction of example 1 (b), thus the object compound was obtained as a colorless solid (1.78 g).

IR(cm⁻¹):3406,2938,1740,1605,1518,1485,1341,1293,1155

MS(m/z):483[M⁺]456,424,373,328,277,247,214,183(BP) 140,115,80,55

¹H-NMR(CDCl₃): 1.76-1.80(1H,m,C5-H),1.93-2.00(1H,m,C5-H),2.15-2.19(1H,m,C3-H), 2.53-2.57(1H,m,C3-H),3.01 (3H,s,Ms),3.31 (1H,dt,J=9.2 Hz,C6-H),3.59(3H,s,CO₂CH₃), 3.87(3H,s,OCH₃),3.94-3.97(1H,m,C6-H),4.71-4.77(1H,m,C4-H),4.93(1H,d,J=6.0 Hz,C2-H), 7.01,7.56(4H,ABq,J=8.8 Hz,aromatic)7.67,7.82(4H,ABq,J=8.4 Hz,aromatic)

(d) (2R,4S)4-Azide-2-methoxycarbonyl-1-[4-(4-methoxyphenyl)benzenesulfonyl]-piperidine

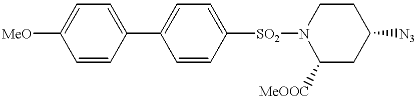

The compound (1.78 g) which was prepared by example 14 (c) was carried out same reaction of example 1 (c), thus the object compound was obtained as a colorless solid (1.45 g).

IR(cm⁻¹):3424,2944,2104,1746,1605,1518,1341,1155

MS(m/z):430[M+]371,247,183(BP)139,95,55

¹H-NMR(CDCl₃): 1.78-1.82(2H,m,C5-H),2.05-2.10,2.52-2.55(2H,eacheachm,C3-H), 3.44(1H,dt,J=9.2 Hz,C6-H),3.62(3H,s,CO₂CH₃),3.69,3.71

(e) (2R,4S)-4-Acetylamino-2-methoxycarbonyl-1-[4-(4-methoxyphenyl)benzene sulfonyl]-piperidine

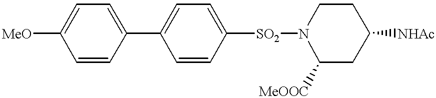

The compound (0.20 g) which was prepared by example 14 (d) was dissolved in pyridine (1.5 mL), then thioacetic acid (1.5 mL) was added at 0° C., and stirred on overnight at room temperature. The reaction mixture was concentrated, then the residue was purified by silica gel column chromatography using ethyl acetate:n-hexane (1:1), the object compound was obtained as a colorless solid (0.19 g).

IR(cm⁻¹):3400,2920,1740,1650,1521,1488,1338,1293,1158

MS(m/z):446[M⁺]381,342,313,281,252,206,175,149(BP) 119,85,55

¹H-NMR(CDCl₃): 1.76-1.84(1H,m,C5-H),1.89(3H,s,Ac),1.99-2.03(1H,m,C5-H), 2.19-2.24(1H,m,C3-H),3.45-3.50(1H,dt,C3-H),3.62(3H,s,CO₂CH₃),3.63-3.74(1H,m,C4-H), 3.87(3H,s,OCH₃),3.85-3.91 (1H,m,C6-H),4.16-4.19(1H,m,C6-H),4.64-4.67(1H,m,C2-H), 6.14-6.17(1H,m,NH),7.01,7.56(4H,ABq,aromatic)7.65,7.85(4H,ABq,aromatic)

(f) (2R,4S)4-Acetylamino-2-carboxy-1-[4-(4-methoxyphenyl)benzenesulfonyl]-piperidine (Compound 44)

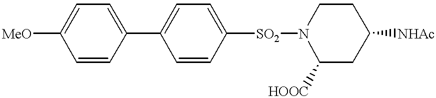

The compound (190 mg) which was prepared by example 14 (e) was carried out same reaction of example 1 (f), thus the object compound was obtained as a colorless solid (160 mg).

IR(cm⁻¹):3412,2914,1725,1608,1338,1251,1155,1092

MS(m/z):433 ┌M⁺+1┘385,328,277,248,216,184,139,108,81 (BP)55

¹H-NMR(CDCl₃): 1.17-1.32(1H,m,C5-H),1.57-1.68(1H,m,C5-H),1.98(3H,s,Ac), 2.39-2.48(1H,m,C3-H),2.86(1H,m,C3-H),3.24-3.30(1H,m,C4-H),3.60(1H,m,C6-H), 3.73-3.76(1H,m,C6-H),3.88(3H,s,OCH₃),4.76(1H,d,J=5.4 Hz,C2-H), 6.99-7.06(2H,m,aromatic)7.52-7.85(6H,m,aromatic),8.40(1H,brm,NH)

Example: 15 (2R,4S)-4-Acetylamino-2-hydroxyaminocarbonyl-1-[4-(4-methoxy-phenyl)benzenesulfonyl]-piperidine (Compound 82)

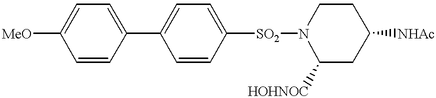

The compound (120 mg) which was prepared by example 14 was carried out same reaction of example 2, thus the object compound was obtained as a colorless solid (68 mg).

IR(cm⁻¹):3424,2914,1638,1488,1335,1251,1152,1092

MS(m/z):447[M⁺]416,371,328,248,216,183,140,115,82(BP)48

¹H-NMR(CD₃OD): 1.11-1.38(2H,m,C5-H),1.84(1H,m,C3-H),1.88(3H,s,Ac), 2.28-2.31 (1H,m,C3-H),3.42-3.49(1H,m,C4-H),3.83(1H,m,C6-H),3.88(3H,s,OCH₃), 3.92(1H,m,C6-H),4.69(1H,d,J=5.4 Hz,C2-H),6.87(1H,d,J=7.3 Hz,NH), 7.02,7.58(4H,ABq,J=8.8 Hz,aromatic)7.72,7.86(4H,ABq,J=8.3 Hz,aromatic)

Example: 16 (2R,4R)-2-Carboxy-1-(4-methoxybenzenesulfonyl)-4-(pyridyl-2-yl) carbonylamino-piperidine (Compound 68)

(a) (2R,4R)-2-Methoxycarbonyl-1-(4-methoxybenzenesulfonyl)-4-(pyridyl-2-yl)carbonyl amino-piperidine

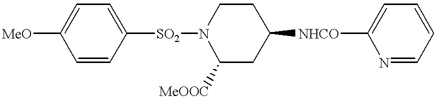

(2R,4S)-4-Amino-2-methoxycarbonyl-1-(4-methoxybenzenesulfonyl)-piperidine (800 mg) was dissolved in DMF (5 mL), then WSCDI (610 mg), Et₃N (0.44 mL), 2-pyridinecarboxylic acid (390 mg), and DMAP (80 mg) were added at 0° C., and stirred for 16 h. The reaction mixture was diluted with ethyl acetate (50 mL), then washed with 10% aq.citric acid, sat.NaHC₃, and brine, respectively. The organic layer was dried (Na₂SO₄), filtered, and concentrated, then the residue was purified by silica gel column chromatography using ethyl acetate:n-hexane (1:1), the object compound was obtained as a colorless oil (900 mg).

IR(cm⁻¹):3364,2938,1734,1662,1593,1458,1338,1254,1149,726

MS(m/z):434[M⁺]374,312,252,208,180,107

¹H-NMR(CDCl₃):1.56(1H,m,C5-H), 1.81 (1H,m,C5-H),2.07(1H,m,C3-H),2.50(1H,m,C3-H), 3.39(1H,dt,J=13.2 Hz,C6-H),3.61(3H,S,CO₂CH₃),3.87(3H,s,OCH₃),3.91 (1H,m,C6-H), 4.05(1H,m,C4-H),4.92(1H,d,J=5.4 Hz,C2-H),6.97(2H,ABq,J=8.8 Hz,aromatic), 7.43(1H,m,pyridine),7.75(2H,ABq,J=8.8 Hz,aromatic),7.84(1H,m,pyridine), 7.90(1H,d,J=7.8 Hz,pyridine),8.15(1H,d,J=7.8 Hz,pyridine),8.54(1H,d,J=4.9 Hz,NH)

(b) (2R,4R)-2—Carboxy-1-(4-methoxybenzenesulfonyl)-4-(pyridyl-2-yl) carbonylamino-piperidine (Compound 68)

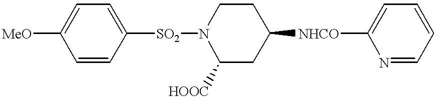

The compound (900 mg) which was prepared by example 16 (a) was carried out same reaction of example 1 (f), thus the object compound was obtained as a colorless solid (820 mg).

IR(cm⁻¹):3340,2932,1728,1590,1326,1266,1095,558

MS(m/z):419[M⁺]310,252,202,171,126,80,52

¹H-NMR(CDCl₃):1.52(1H,m,C5-H), 1.80(1H,m,C5-H), 1.97(1H,m,C3-H),2.55(1H,m,C3-H), 3.31 (1H,dt,J=13.2 Hz,C6-H),3.75 (1H,m,C6-H),3.83(3H,s,OCH₃),4.20(1H,m,C4-H), 4.93(1H,d,J=5.4 Hz,C2-H),6.93(2H,ABq,J=9.3 Hz,aromatic),7.47(1H,m,pyridine), 7.77(2H,ABq,J=9.3 Hz,aromatic),7.86(1H,dt,J=7.8,7.3 Hz,pyridine),8.14(1H,m,pyridine), 8.55(1H,d,J=8.8 Hz,NH)

Example: 17 (2R,4R)-2-Hydroxyaminocarbonyl-1-(4-methoxybenzene sulfonyl)-4-(pyridyl-2-yl)carbonylamino-piperidine (Compound 69)

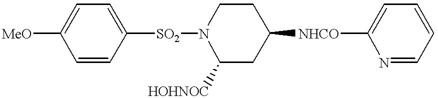

The compound (160 mg) which was prepared by example 16 was carried out same reaction of example 2, thus the object compound was obtained as a colorless solid (90 mg).

IR(cm⁻¹):3214,2908,1656,1524,1461,1335,1257,1149,1023,558

MS(m/z):434[M⁺]413,374,341,284,253,204,177,157,131,99,66

¹H-NMR(CD₃OD):1.48(2H,m,C5-H),2.00(1H,m,C3-H),2.40(1H,m,C3-H),3.54(1H,m,C6-H), 3.80(3H,s,OCH₃),3.94(1H,m,C6-H),4.30(1H,m,C4-H),4.73(1H,d,J=5.4 Hz,C2-H), 6.97(2H,ABq,J=8.8 Hz,aromatic),7.42(1H,m,pyridine),7.77(2H,ABq,J=8.8 Hz,aromatic), 7.84(1H,t,J=7.8 Hz,pyridine),8.18(1H,d,J=7.8 Hz,pyridine),8.50(1H,d,J=4.4 Hz,pyridine), 10.31(1H,s,OH)

| Comp. No. | m.p. (° C.) |
|---|---|
| 1 | 147˜149 |
| 2 | 95˜97 |
| 3 | 240˜243 |
| 4 | >250 |
| 5 | 102˜104 |
| 6 | 205˜210 |
| 7 | 72˜73 |
| 8 | 185˜186 |
| 9 | 233˜235 |
| 10 | 150˜151 |
| 11 | 42˜43 |
| 12 | 84˜85 |
| 13 | 61˜62 |
| 14 | >250 |
| 15 | oil |
| 16 | 164˜166 |
| 17 | 228˜230 |
| 18 | 60˜62 |
| 19 | 254˜256 |
| 20 | >250 |
| 21 | 235˜238 |
| 22 | 210˜211 |
| 23 | 170˜172 |
| 24 | 90˜92 |
| 25 | 120˜121 |
| 26 | 149˜150 |
| 27 | >250 |
| 28 | 252˜253 |
| 29 | 238˜239 |
| 30 | 185˜186 |
| 31 | 243˜245 |
| 32 | 245˜246 |
| 33 | 226˜227 |
| 34 | 228˜230 |
| 35 | 193˜195 |
| 36 | 258˜259 |
| 37 | 237˜238 |
| 38 | 152˜154 |
| 39 | 220˜223 |
| 40 | 100˜102 |
| 41 | 150˜152 |
| 42 | 240˜244 |
| 43 | 94˜96 |
| 44 | 148˜149 |
| 45 | 82˜84 |
| 46 | 60˜62 |
| 47 | 176˜178 |
| 48 | 181˜182 |
| 49 | 106˜108 |
| 50 | 130˜135 |
| 51 | 93˜95 |
| 52 | 85˜86 |
| 53 | 90˜91 |
| 54 | 88˜89 |
| 55 | 94˜96 |
| 56 | 158˜160 |
| 57 | 88˜90 |
| 58 | 170˜172 |
| 59 | 88˜90 |
| 60 | 160˜162 |
| 61 | 91˜93 |
| 62 | 144˜146 |
| 63 | 125˜129 |
| 64 | 178˜179 |
| 65 | 155˜156 |
| 66 | 118˜120 |
| 67 | 110˜112 |
| 68 | 139˜141 |
| 69 | 99˜100 |
| 70 | 131˜132 |
| 71 | 113˜114 |
| 72 | 188˜189 |
| 73 | 135˜137 |
| 74 | 145˜148 |
| 75 | 164˜165 |
| 76 | 177˜179 |
| 77 | 177˜178 |
| 78 | 106˜108 |
| 79 | 135˜136 |
| 80 | 190˜192 |
| 81 | 108˜110 |
| 82 | 198˜199 |
| 83 | 162˜163 |
| 84 | 110˜111 |
| 85 | 153˜154 |
| 86 | 183˜184 |
| 87 | 243˜245 |
| 88 | 180˜182 |
| 89 | 239˜240 |

What is claimes is:

1. A compound of general formula (I) or a pharmaceutically permittable salt thereof. 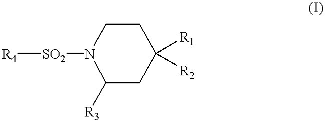
wherein, in case of R₁ and R₂ are same functional groups, it represents ═O, ═N—OR₉ (R₉ is hydrogen, lower alkyl, or benzyl). And, in the other case, one of R₁ and R₂ represents hydrogen, and another one represents —R₅-R₆ (R₅ is —O—, —NH—, —NHCO—, or —NHSO₂—. R₆ is hydrogen, lower alkyl, —Ph—R₇, —(CH₂)n—O—Ph—R₇ (n=1-6, R₇ is hydrogen, hydroxy, lower alkyl, lower alkoxy, halogen, nitro, or pyridyloxy), indolyl, N-oxidepyridyl, phtalimide, thienyl, or pyridyl).
R₃ represents —COOH, —COOEt, —COOMe, —CH₂N(OH)CHO, or —CONHOH.
R₄ represnts lower alkyl, thienyl, —Ph—R₈ (R₈ represents hydroxy, lower alkyl, lower alkoxy, nitro, halogen, pyridyloxy, or phenyl group substituted hydrogen, lower alkyl, lower alkoxy, hydroxy, and halogen).

2. A method for preparing a compound of general formula (III), 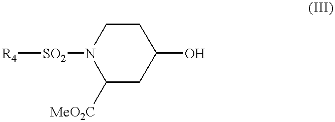
(wherein R₄ is the same as mentioned above.)
comprising reacting a compound of general formula (II), 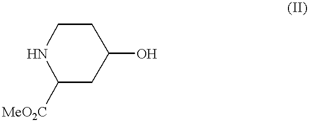
with a compound that represents R₄SO₂Cl (wherein R₄ is the same as mentioned above) in presence of base to produce the compound of general formula (III).

3. A method for preparing a compound of general formula (VI), 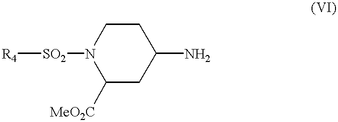
(wherein R₄ is the same as mentioned above.)
comprising reacting a compound of general formula (III) with MsCl in presence of base to yield the compound of general formula (IV), 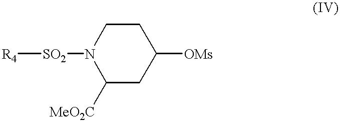
(wherein R₄ is the same as mentioned above.)
then, it is carried out azidation to produce the compound of general formula (V). 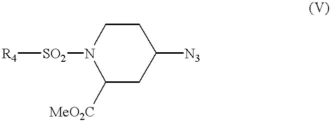
(wherein R₄ is the same as mentioned above.)
Subsecuently, azide group of the compound (V) is reduced to yield the compound of general formula (VI).

4. A method for preparing a compound of general formula (VII), 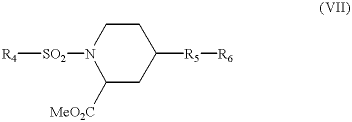
(wherein R₄ and R₆ are the same as mentioned above, R₅ is —NH—, —NHCO—, or —NHSO₂—)
comprising reacting a compound of general formula (VI) with R₆COOH (wherein R₆ is the same as mentioned above.), R₆SO₂Cl (wherein R₆ is the same as mentioned above) in presence of base, or reductive aminetion using R₆CHO (wherein R₆ is the same as mentioned above) to produce the compound of general formula (VII).

5. A method for preparing a compound of general formula (VIII), 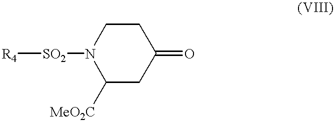
(wherein R₄ is the same as mentioned above.)
comprising reacting a compound of general formula (III) with oxidant to produce the compound of general formula (VIII).

6. A method for preparing a compound of general formula (IX), 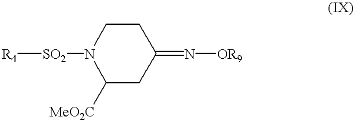
(wherein R₄ and R₉ are the same as mentioned above.)
comprising reacting a compound of general formula (VIII) with R₉₀—NH₂ HCl to produce the compound of general formula (IX).

7. A method for preparing a compound of general formula (XI), 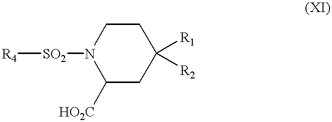
(wherein R₁, R₂, and R₄ are the same as mentioned above.)
comprising hydrolyzing a compound of general formula (X), 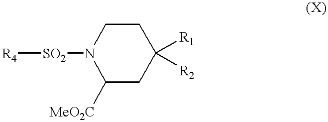
(wherein R₁, R₂, and R₄ are the same as mentioned above.)
to produce the compound of general formula (XI).

8. A method for preparing a compound of general formula (XII), 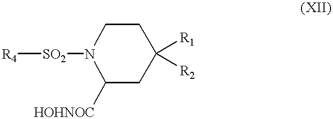
(wherein R₁, R₂, and R₄ are the same as mentioned above.)
comprising reacting a compound of general formula (X) or (XI) with HONH₂ HCl to produce the compound of general formula (XII).

9. A method for preparing a compound of general formula (XVII), 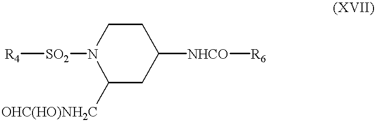
(wherein R₄, and R₆ are the same as mentioned above.)
comprising reacting a compound of general formula (XIII), 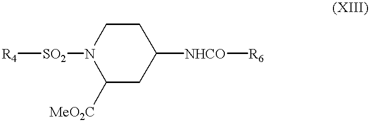
(wherein R₄, and R₆ are the same as mentioned above.)
with reductant to produce the compound of general formula (XIV), 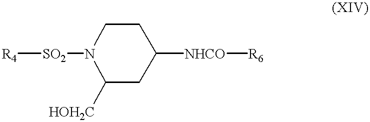
(wherein R₄, and R₆ are the same as mentioned above.)
then it is oxidazed to yield the compound of general formula (XV). 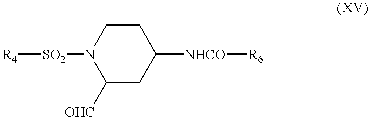
(wherein R₄, and R₆ are the same as mentioned above.)
Subsecuently, it is carried out reductive amination to yield the compound of general formula (XVI), 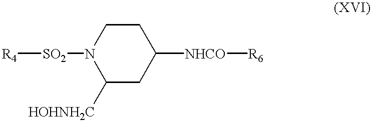
(wherein R₄, and R₆ are the same as mentioned above.)
then it is carried out formylation to produce the compound of general formula (XVII).

10. A phramaceutical composition that are used to treatment and/or prevention for disease related to destruction of extra cellular matrix induced by a matrix metalloproteinases, wherein an active ingredient is a compound of the general formula (I) or a salt thereof with pharmaceutically acceptable base.
